(19) United States
(12) Patent Application Publication
(10) Pub. No.: US 2025/0129051 A1
(43) Pub. Date: Apr. 24, 2025

(54) PARP1 INHIBITORS AND USES THEREOF

(71) Applicant: Gilead Sciences, Inc., Foster City, CA (US)

(72) Inventors: Robert L. Hoffman, San Diego, CA (US); Michael R. Mish, Foster City, CA (US); James G. Taylor, Burlingame, CA (US)

(21) Appl. No.: 18/922,134

(22) Filed: Oct. 21, 2024

Related U.S. Application Data
(60) Provisional application No. 63/592,458, filed on Oct. 23, 2023.

(51) Int. Cl. C07D 401/14; A61K 31/444; A61K 31/4709; A61K 31/498; A61P 35/00; C07D 471/04; C07D 491/048

(52) U.S. Cl. CPC C07D 401/14; A61K 31/444; A61K 31/4709; A61K 31/498; A61P 35/00; C07D 471/04; C07D 491/048

(57) ABSTRACT

Described herein are PARP1 inhibitors and pharmaceutical compositions comprising said inhibitors. The subject compounds and compositions are useful for the treatment of cancer.

### CROSS REFERENCE TO RELATED APPLICATION

This application claims the benefit of U.S. Provisional Application No. 63/592,458, filed Oct. 23, 2023, which is hereby incorporated by reference in its entirety.

### BACKGROUND

Poly(ADP-ribose) polymerase (PARP) or poly(ADP-ribose) synthase (PARS) has an essential role in facilitating DNA repair, controlling RNA transcription, mediating cell death, and regulating immune response. These actions make PARP inhibitors targets for a broad spectrum of disorders. PARP inhibitors have demonstrated efficacy in numerous models of disease, particularly in models of ischemia reperfusion injury, inflammatory disease, degenerative diseases, protection from adverse effects of cytotoxic compounds, and the potentiation of cytotoxic cancer therapy. PARP has also been indicated in retroviral infection and thus inhibitors may have use in antiretroviral therapy. PARP inhibitors have been efficacious in preventing ischemia reperfusion injury in models of myocardial infarction, stroke, other neural trauma, organ transplantation, as well as reperfusion of the eye, kidney, gut, and skeletal muscle. Inhibitors have been efficacious in inflammatory diseases such as arthritis, gout, inflammatory bowel disease, CNS inflammation such as MS and allergic encephalitis, sepsis, septic shock, hemorrhagic shock, pulmonary fibrosis, and uveitis. PARP inhibitors have also shown benefit in several models of degenerative disease including diabetes (as well as complications) and Parkinson's disease. PARP inhibitors can ameliorate the liver toxicity following acetaminophen overdose, cardiac and kidney toxicities from doxorubicin and platinum based antineoplastic agents, as well as skin damage secondary to sulfur mustards. In various cancer models, PARP inhibitors have been shown to potentiate radiation and chemotherapy by increasing cell death of cancer cells, limiting tumor growth, decreasing metastasis, and prolonging the survival of tumor-bearing animals.

PARP1 and PARP2 are the most extensively studied PARPs for their role in DNA damage repair. PARP1 is activated by DNA damage breaks and functions to catalyze the addition of poly(ADP-ribose) (PAR) chains to target proteins. This post-translational modification, known as PARylation, mediates the recruitment of additional DNA repair factors to DNA lesions.

Following completion of this recruitment role, PARP auto-PARylation triggers the release of bound PARP from DNA to allow access to other DNA repair proteins to complete repair. Thus, the binding of PARP to damaged sites, its catalytic activity, and its eventual release from DNA are all important steps for a cancer cell to respond to DNA damage caused by chemotherapeutic agents and radiation therapy.

Inhibition of PARP family enzymes has been exploited as a strategy to selectively kill cancer cells by inactivating complementary DNA repair pathways. A number of pre-clinical and clinical studies have demonstrated that tumor cells bearing deleterious alterations of BRCA1 or BRCA2, key tumor suppressor proteins involved in double-strand DNA break (DSB) repair by homologous recombination (HR), are selectively sensitive to small molecule inhibitors of the PARP family of DNA repair enzymes. Such tumors have deficient homologous recombination repair (HRR) pathways and are dependent on PARP enzymes function for survival. Although PARP inhibitor therapy has predominantly targeted SRCA-mutated cancers, PARP inhibitors have been tested clinically in non-SRCA-mutant tumors, those which exhibit homologous recombination deficiency (HRD).

It is believed that PARP inhibitors having improved selectivity for PARP1 may possess improved efficacy and reduced toxicity compared to other clinical PARP1/2 inhibitors. It is believed also that selective strong inhibition of PARP1 would lead to trapping of PARP1 on DNA, resulting in DNA double strand breaks (DSBs) through collapse of replication forks in S-phase. It is believed also that PARP1-DNA trapping is an effective mechanism for selectively killing tumor cells having HRD. An unmet medical need therefore exists for effective and safe PARP inhibitors, especially PARP inhibitors having selectivity for PARP1.

### SUMMARY

Provided herein is a compound which is:

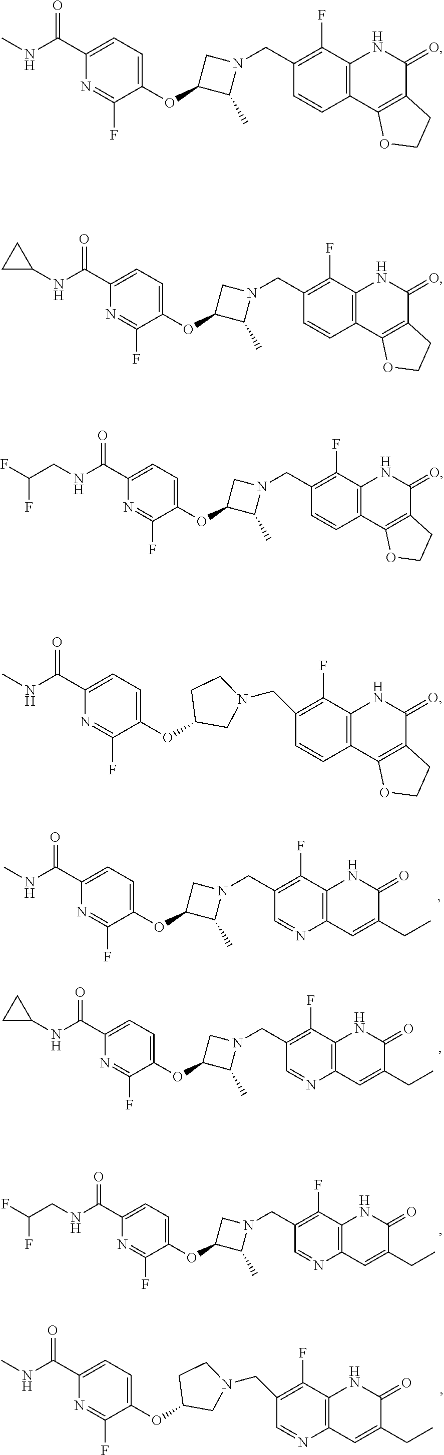

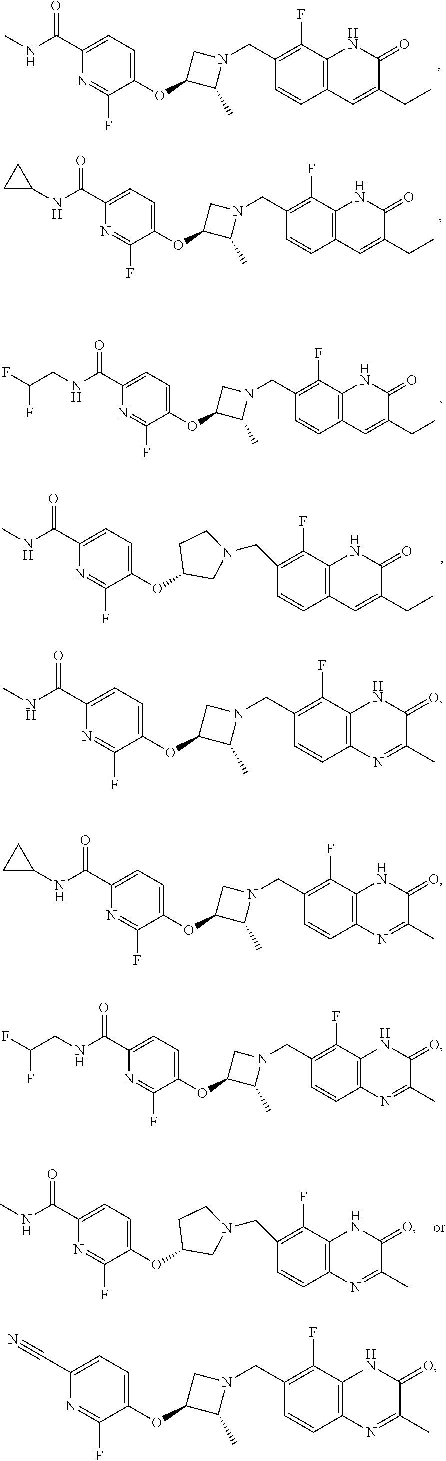

or a pharmaceutically acceptable salt, solvate, or stereoisomer thereof.

Also disclosed herein is a pharmaceutical composition comprising a compound disclosed herein, or a pharmaceutically acceptable salt, solvate, or stereoisomer thereof, and a pharmaceutically acceptable excipient.

Also disclosed herein is a method of treating cancer in a subject in need thereof, the method comprising administering a therapeutically effective amount of a compound disclosed herein, or a pharmaceutically acceptable salt, solvate, or stereoisomer thereof. Also disclosed herein is a method of treating a cancer comprising a BRCA1 and/or a BRCA2 mutation in a subject in need thereof, the method comprising administering a compound disclosed herein, or a pharmaceutically acceptable salt, solvate, or stereoisomer thereof. Also disclosed herein is a method of treating a cancer comprising a mutation in a gene conferring homologous repair deficiency in a subject in need thereof, the method comprising administering a compound disclosed herein, or a pharmaceutically acceptable salt, solvate, or stereoisomer thereof. In some embodiments, the mutation in a gene conferring homologous repair deficiency comprises ATM, BRCA1, BRCA2, BARD1, BRIP1, CDK12, CHEK1, CHEK2, FANCL, PALB2, RAD51B, RAD51C, RAD51D, or RAD54L, or any combinations thereof. In some embodiments, the cancer is bladder cancer, brain & CNS cancer, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer. In some embodiments, the cancer is breast cancer, ovarian cancer, pancreatic cancer, prostate cancer, a hematological cancer, gastrointestinal cancer, or lung cancer. In some embodiments, the cancer is metastatic cancer. In some embodiments, the cancer has metastasized in the brain.

Also disclosed herein is a method of treating a cancer that is present in the brain in a subject in need thereof, the method comprising administering a compound disclosed herein, or a pharmaceutically acceptable salt, solvate, or stereoisomer thereof.

Also disclosed herein is a method of treating brain cancer in a subject in need thereof, the method comprising administering a therapeutically effective amount of a compound disclosed herein, or a pharmaceutically acceptable salt, solvate, or stereoisomer thereof. In some embodiments, the compound or a pharmaceutically acceptable salt, solvate, or stereoisomer thereof, is brain penetrant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the disclosure.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

As used herein, a “disease or disorder associated with PARP” or, alternatively, “a PARP-mediated disease or disorder” means any disease or other deleterious condition in which PARP, or a mutant thereof, is known or suspected to play a role.

As used herein, a “disease or disorder associated with PARP1” or, alternatively, “a PARP1-mediated disease or disorder” means any disease or other deleterious condition in which PARP1, or a mutant thereof, is known or suspected to play a role.

### Compounds

Described herein are compounds, or a pharmaceutically acceptable salt, solvate, or stereoisomer thereof, useful in the treatment of cancer.

Some embodiments provide for a compound which is:

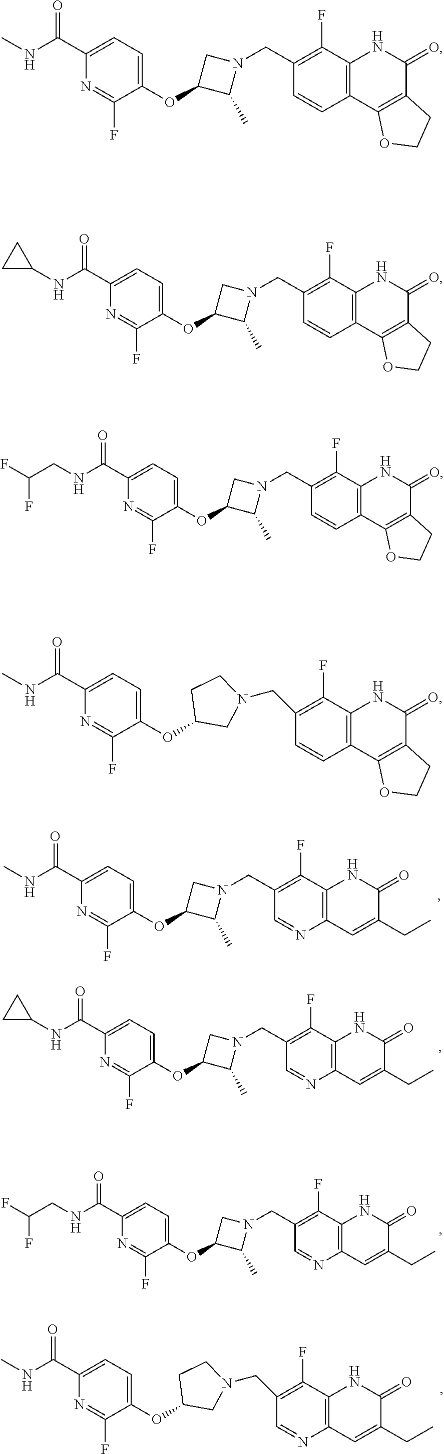

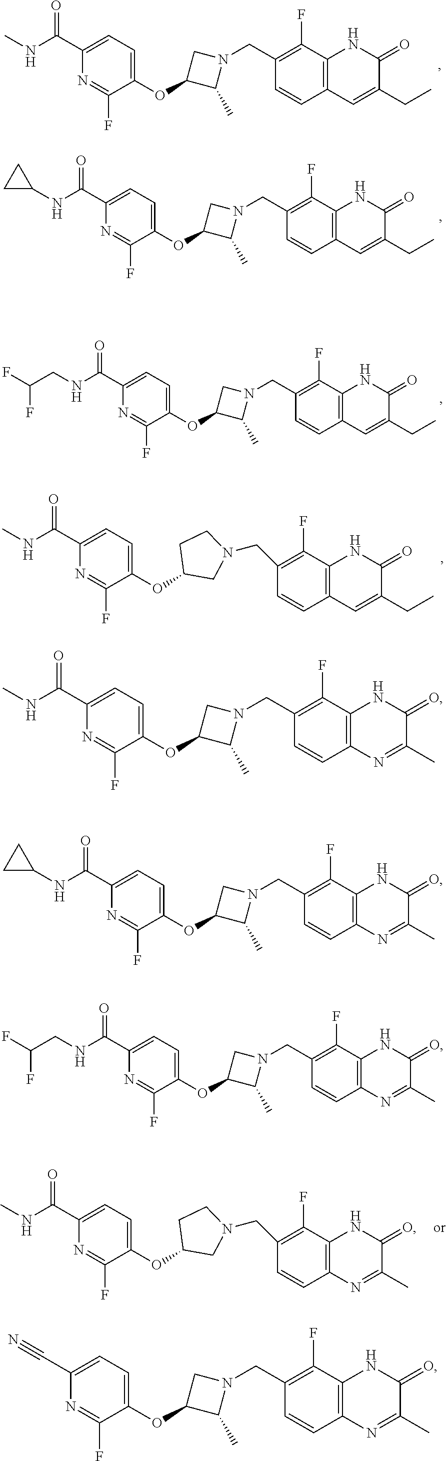

or a pharmaceutically acceptable salt thereof.

Some embodiments provide for a compound which is:

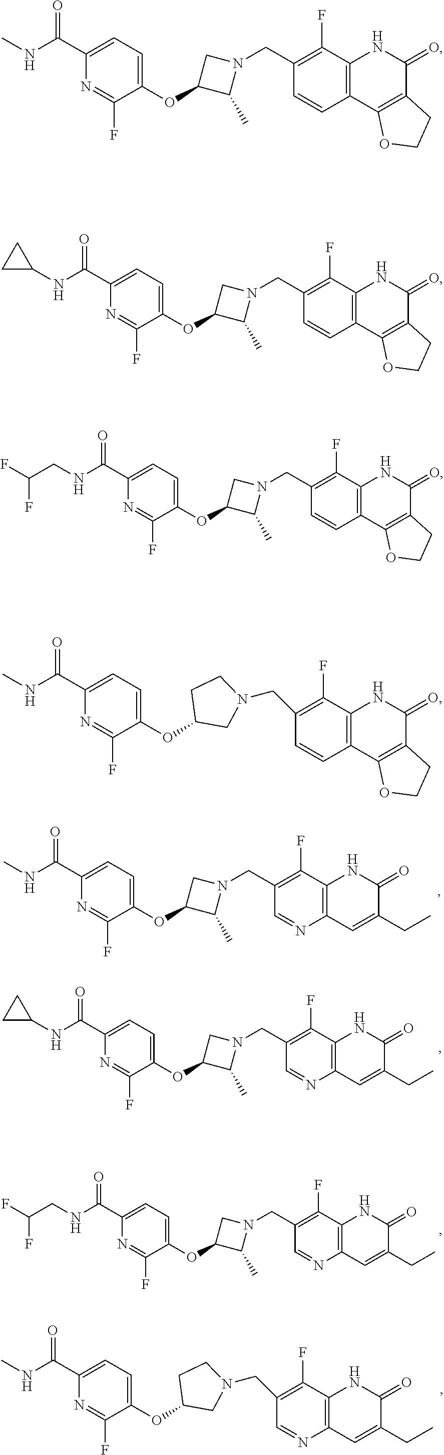

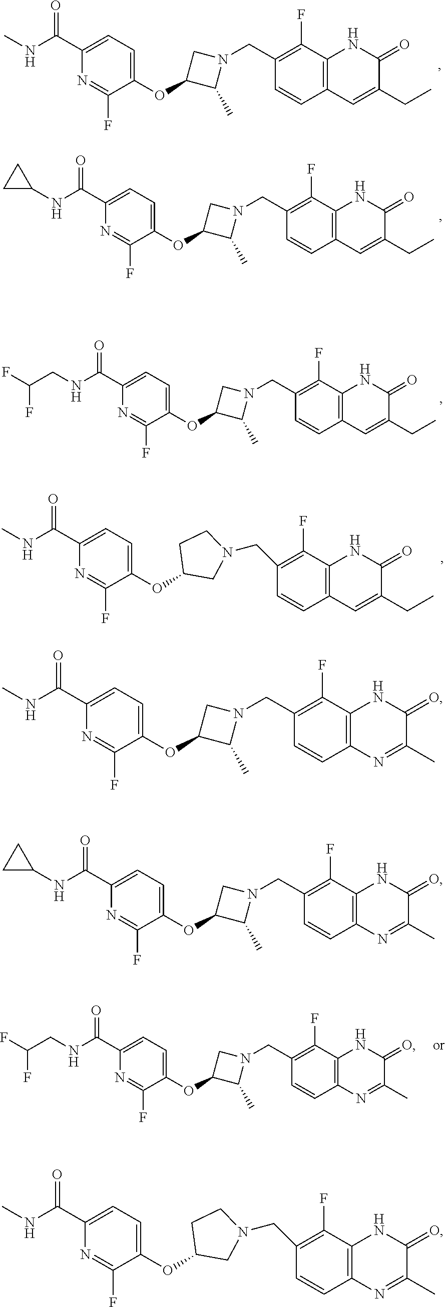

or a pharmaceutically acceptable salt thereof.

In some embodiments, the compound, or a pharmaceutically acceptable salt thereof, is brain penetrant.

Disclosed herein are a compound, or pharmaceutically acceptable salt, solvate, or stereoisomer thereof, selected from Table 1.

TABLE 1

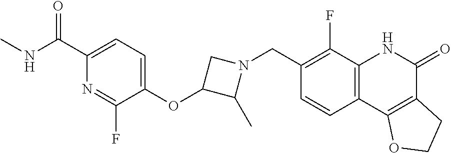 [SMILES: CNC(=O)c1ccc(OC2CN(Cc3ccc4c5c(c(=O)[nH]c4c3F)CCO5)C2C)c(F)n1]

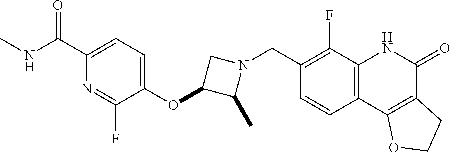 [SMILES: CNC(=O)c1ccc(O[C@H]2CN(Cc3ccc4c5c(c(=O)[nH]c4c3F)CCO5)[C@H]2C)c(F)n1]

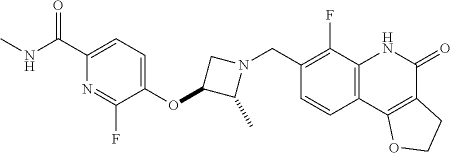 [SMILES: CNC(=O)c1ccc(O[C@H]2CN(Cc3ccc4c5c(c(=O)[nH]c4c3F)CCO5)[C@@H]2C)c(F)n1]

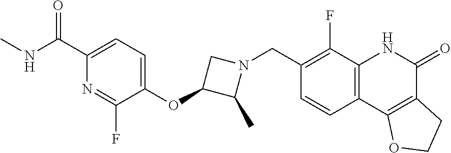 [SMILES: CNC(=O)c1ccc(O[C@H]2CN(Cc3ccc4c5c(c(=O)[nH]c4c3F)CCO5)[C@H]2C)c(F)n1]

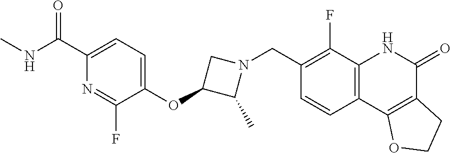 [SMILES: CNC(=O)c1ccc(O[C@H]2CN(Cc3ccc4c5c(c(=O)[nH]c4c3F)CCO5)[C@@H]2C)c(F)n1]

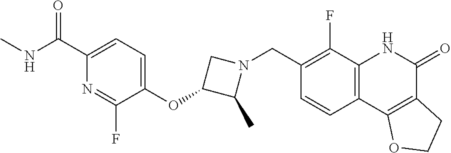 [SMILES: CNC(=O)c1ccc(O[C@@H]2CN(Cc3ccc4c5c(c(=O)[nH]c4c3F)CCO5)[C@H]2C)c(F)n1]

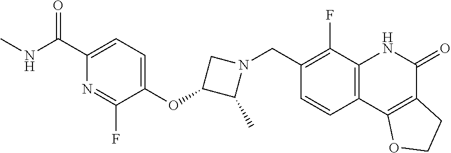 [SMILES: CNC(=O)c1ccc(O[C@@H]2CN(Cc3ccc4c5c(c(=O)[nH]c4c3F)CCO5)[C@@H]2C)c(F)n1]

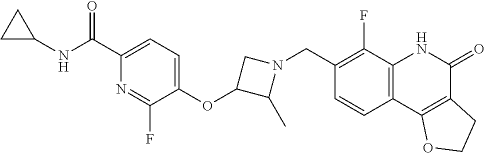 [SMILES: CC1C(Oc2ccc(C(=O)NC3CC3)nc2F)CN1Cc1ccc2c3c(c(=O)[nH]c2c1F)CCO3]

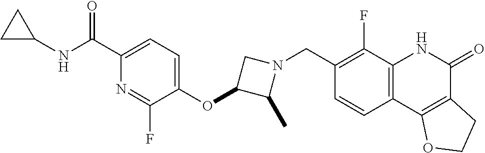 [SMILES: C[C@H]1[C@@H](Oc2ccc(C(=O)NC3CC3)nc2F)CN1Cc1ccc2c3c(c(=O)[nH]c2c1F)CCO3]

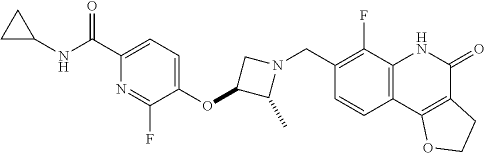 [SMILES: C[C@@H]1[C@@H](Oc2ccc(C(=O)NC3CC3)nc2F)CN1Cc1ccc2c3c(c(=O)[nH]c2c1F)CCO3]

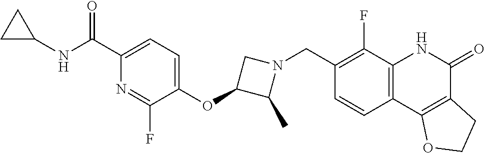 [SMILES: C[C@H]1[C@@H](Oc2ccc(C(=O)NC3CC3)nc2F)CN1Cc1ccc2c3c(c(=O)[nH]c2c1F)CCO3]

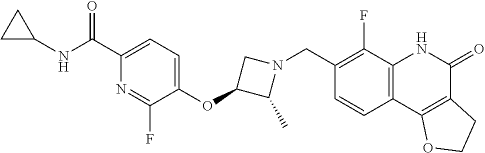 [SMILES: C[C@@H]1[C@@H](Oc2ccc(C(=O)NC3CC3)nc2F)CN1Cc1ccc2c3c(c(=O)[nH]c2c1F)CCO3]

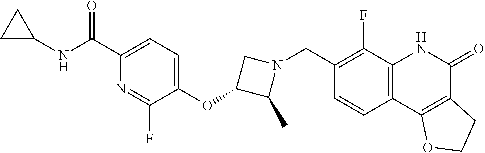 [SMILES: C[C@H]1[C@H](Oc2ccc(C(=O)NC3CC3)nc2F)CN1Cc1ccc2c3c(c(=O)[nH]c2c1F)CCO3]

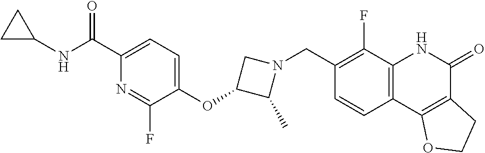 [SMILES: C[C@@H]1[C@H](Oc2ccc(C(=O)NC3CC3)nc2F)CN1Cc1ccc2c3c(c(=O)[nH]c2c1F)CCO3]

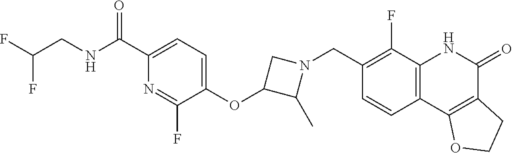 [SMILES: CC1C(Oc2ccc(C(=O)NCC(F)F)nc2F)CN1Cc1ccc2c3c(c(=O)[nH]c2c1F)CCO3]

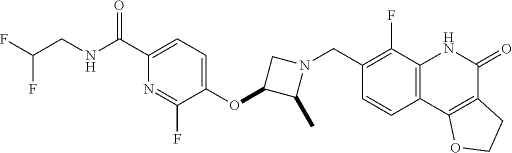 [SMILES: C[C@H]1[C@@H](Oc2ccc(C(=O)NCC(F)F)nc2F)CN1Cc1ccc2c3c(c(=O)[nH]c2c1F)CCO3]

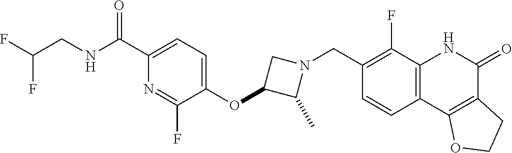 [SMILES: C[C@@H]1[C@@H](Oc2ccc(C(=O)NCC(F)F)nc2F)CN1Cc1ccc2c3c(c(=O)[nH]c2c1F)CCO3]

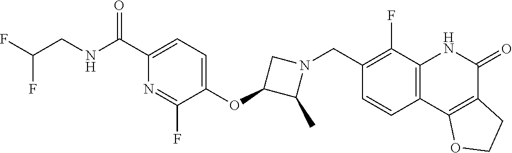 [SMILES: C[C@H]1[C@@H](Oc2ccc(C(=O)NCC(F)F)nc2F)CN1Cc1ccc2c3c(c(=O)[nH]c2c1F)CCO3]

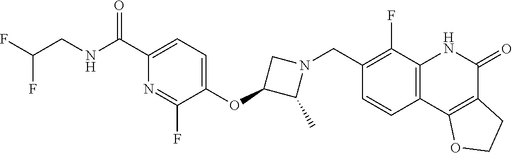 [SMILES: C[C@@H]1[C@@H](Oc2ccc(C(=O)NCC(F)F)nc2F)CN1Cc1ccc2c3c(c(=O)[nH]c2c1F)CCO3]

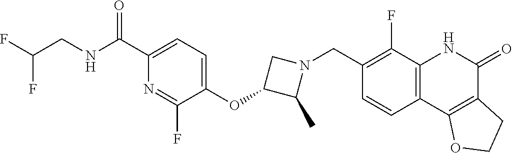 [SMILES: C[C@H]1[C@H](Oc2ccc(C(=O)NCC(F)F)nc2F)CN1Cc1ccc2c3c(c(=O)[nH]c2c1F)CCO3]

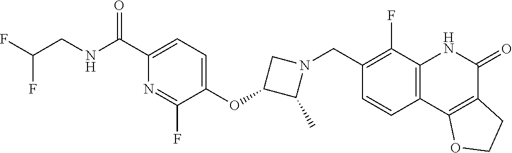 [SMILES: C[C@@H]1[C@H](Oc2ccc(C(=O)NCC(F)F)nc2F)CN1Cc1ccc2c3c(c(=O)[nH]c2c1F)CCO3]

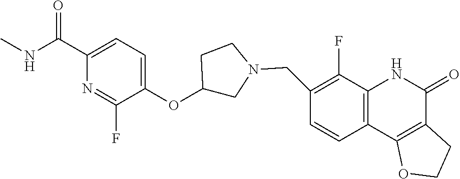 [SMILES: CNC(=O)c1ccc(OC2CCN(Cc3ccc4c5c(c(=O)[nH]c4c3F)CCO5)C2)c(F)n1]

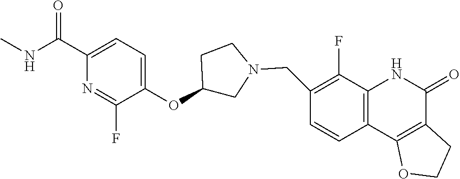 [SMILES: CNC(=O)c1ccc(O[C@H]2CCN(Cc3ccc4c5c(c(=O)[nH]c4c3F)CCO5)C2)c(F)n1]

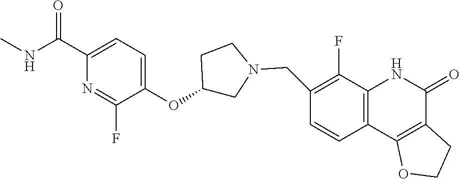 [SMILES: CNC(=O)c1ccc(O[C@@H]2CCN(Cc3ccc4c5c(c(=O)[nH]c4c3F)CCO5)C2)c(F)n1]

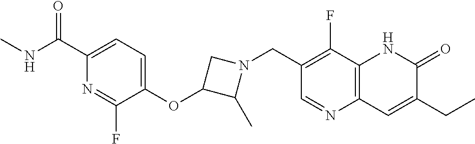 [SMILES: CCc1cc2ncc(CN3CC(Oc4ccc(C(=O)NC)nc4F)C3C)c(F)c2[nH]c1=O]

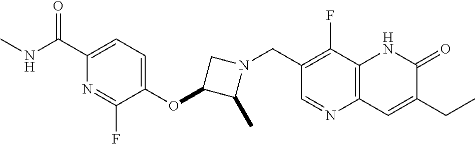 [SMILES: CCc1cc2ncc(CN3C[C@H](Oc4ccc(C(=O)NC)nc4F)[C@@H]3C)c(F)c2[nH]c1=O]

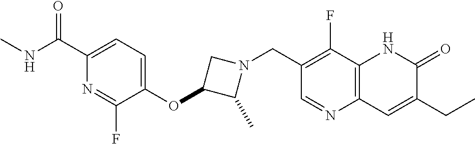 [SMILES: CCc1cc2ncc(CN3C[C@H](Oc4ccc(C(=O)NC)nc4F)[C@H]3C)c(F)c2[nH]c1=O]

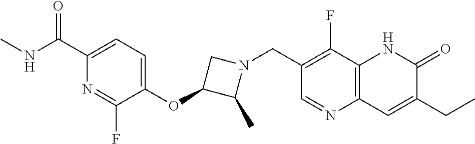 [SMILES: CCc1cc2ncc(CN3C[C@H](Oc4ccc(C(=O)NC)nc4F)[C@@H]3C)c(F)c2[nH]c1=O]

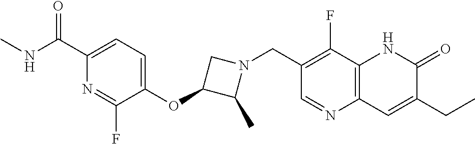 [SMILES: CCc1cc2ncc(CN3C[C@H](Oc4ccc(C(=O)NC)nc4F)[C@@H]3C)c(F)c2[nH]c1=O]

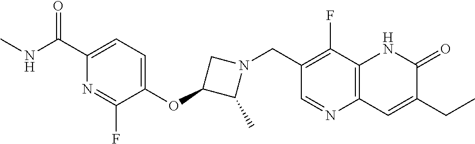 [SMILES: CCc1cc2ncc(CN3C[C@H](Oc4ccc(C(=O)NC)nc4F)[C@H]3C)c(F)c2[nH]c1=O]

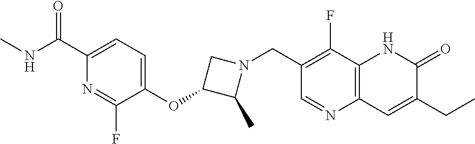 [SMILES: CCc1cc2ncc(CN3C[C@@H](Oc4ccc(C(=O)NC)nc4F)[C@@H]3C)c(F)c2[nH]c1=O]

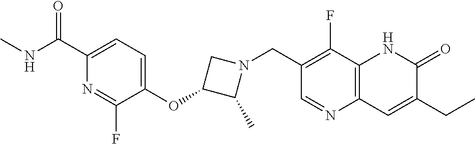 [SMILES: CCc1cc2ncc(CN3C[C@@H](Oc4ccc(C(=O)NC)nc4F)[C@H]3C)c(F)c2[nH]c1=O]

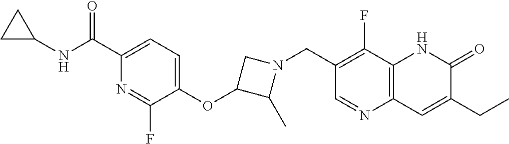 [SMILES: CCc1cc2ncc(CN3CC(Oc4ccc(C(=O)NC5CC5)nc4F)C3C)c(F)c2[nH]c1=O]

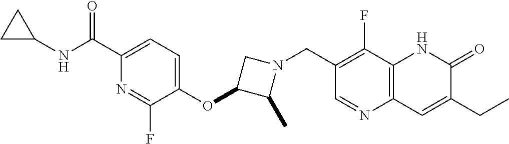 [SMILES: CCc1cc2ncc(CN3C[C@H](Oc4ccc(C(=O)NC5CC5)nc4F)[C@@H]3C)c(F)c2[nH]c1=O]

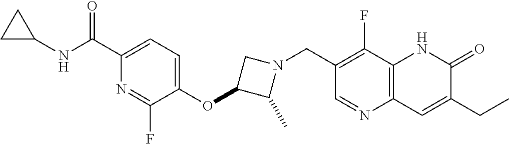 [SMILES: CCc1cc2ncc(CN3C[C@H](Oc4ccc(C(=O)NC5CC5)nc4F)[C@H]3C)c(F)c2[nH]c1=O]

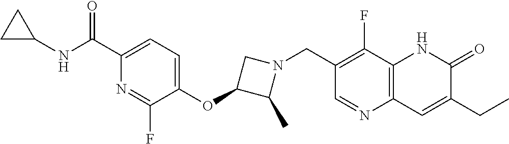 [SMILES: CCc1cc2ncc(CN3C[C@H](Oc4ccc(C(=O)NC5CC5)nc4F)[C@@H]3C)c(F)c2[nH]c1=O]

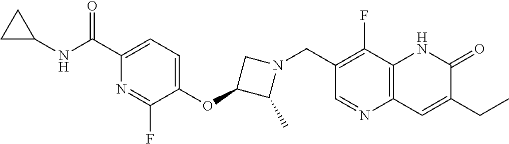 [SMILES: CCc1cc2ncc(CN3C[C@H](Oc4ccc(C(=O)NC5CC5)nc4F)[C@H]3C)c(F)c2[nH]c1=O]

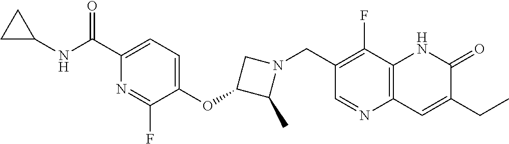 [SMILES: CCc1cc2ncc(CN3C[C@@H](Oc4ccc(C(=O)NC5CC5)nc4F)[C@@H]3C)c(F)c2[nH]c1=O]

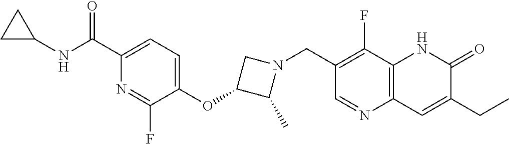 [SMILES: CCc1cc2ncc(CN3C[C@@H](Oc4ccc(C(=O)NC5CC5)nc4F)[C@H]3C)c(F)c2[nH]c1=O]

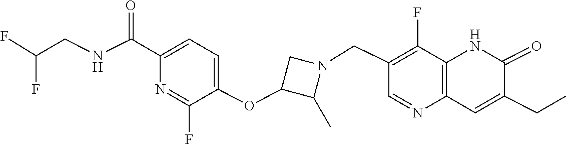 [SMILES: CCc1cc2ncc(CN3CC(Oc4ccc(C(=O)NCC(F)F)nc4F)C3C)c(F)c2[nH]c1=O]

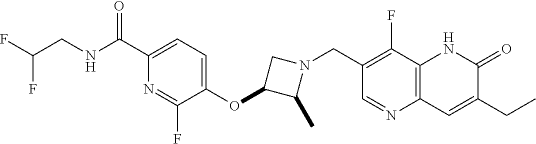 [SMILES: CCc1cc2ncc(CN3C[C@H](Oc4ccc(C(=O)NCC(F)F)nc4F)[C@@H]3C)c(F)c2[nH]c1=O]

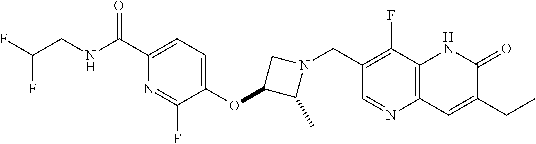 [SMILES: CCc1cc2ncc(CN3C[C@H](Oc4ccc(C(=O)NCC(F)F)nc4F)[C@H]3C)c(F)c2[nH]c1=O]

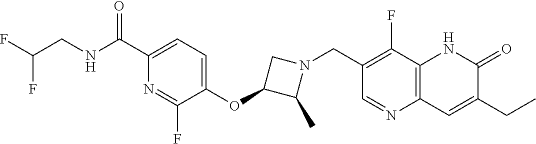 [SMILES: CCc1cc2ncc(CN3C[C@H](Oc4ccc(C(=O)NCC(F)F)nc4F)[C@@H]3C)c(F)c2[nH]c1=O]

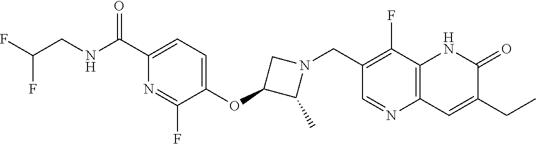 [SMILES: CCc1cc2ncc(CN3C[C@H](Oc4ccc(C(=O)NCC(F)F)nc4F)[C@H]3C)c(F)c2[nH]c1=O]

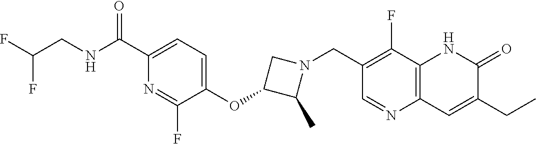 [SMILES: CCc1cc2ncc(CN3C[C@@H](Oc4ccc(C(=O)NCC(F)F)nc4F)[C@@H]3C)c(F)c2[nH]c1=O]

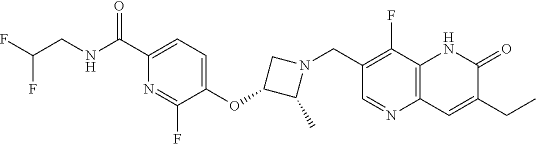 [SMILES: CCc1cc2ncc(CN3C[C@@H](Oc4ccc(C(=O)NCC(F)F)nc4F)[C@H]3C)c(F)c2[nH]c1=O]

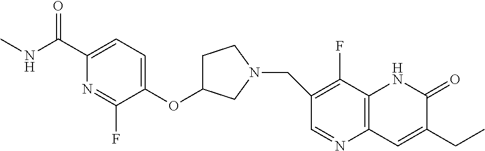 [SMILES: CCc1cc2ncc(CN3CCC(Oc4ccc(C(=O)NC)nc4F)C3)c(F)c2[nH]c1=O]

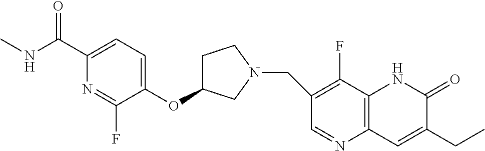 [SMILES: CCc1cc2ncc(CN3CC[C@H](Oc4ccc(C(=O)NC)nc4F)C3)c(F)c2[nH]c1=O]

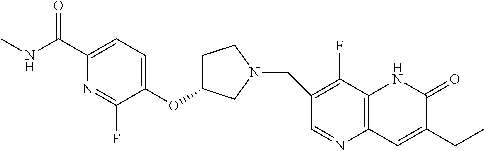 [SMILES: CCc1cc2ncc(CN3CC[C@@H](Oc4ccc(C(=O)NC)nc4F)C3)c(F)c2[nH]c1=O]

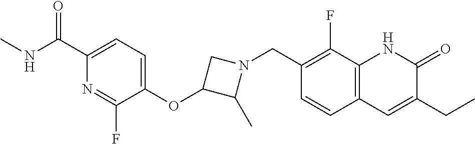 [SMILES: CCc1cc2ccc(CN3CC(Oc4ccc(C(=O)NC)nc4F)C3C)c(F)c2[nH]c1=O]

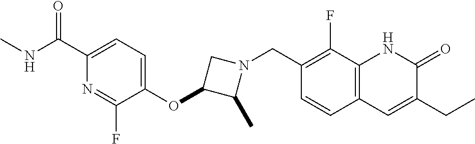 [SMILES: CCc1cc2ccc(CN3C[C@H](Oc4ccc(C(=O)NC)nc4F)[C@@H]3C)c(F)c2[nH]c1=O]

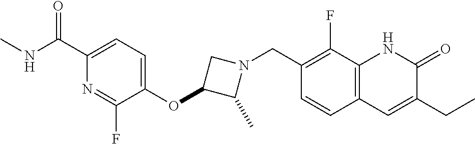 [SMILES: CCc1cc2ccc(CN3C[C@H](Oc4ccc(C(=O)NC)nc4F)[C@H]3C)c(F)c2[nH]c1=O]

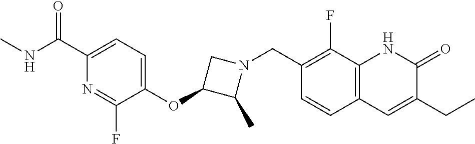 [SMILES: CCc1cc2ccc(CN3C[C@H](Oc4ccc(C(=O)NC)nc4F)[C@@H]3C)c(F)c2[nH]c1=O]

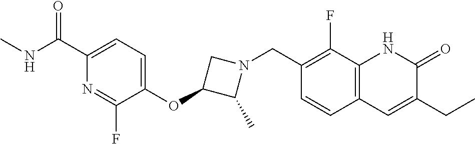 [SMILES: CCc1cc2ccc(CN3C[C@H](Oc4ccc(C(=O)NC)nc4F)[C@H]3C)c(F)c2[nH]c1=O]

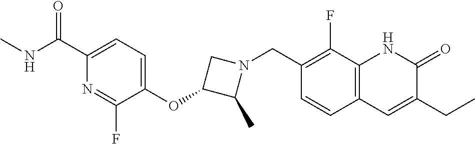 [SMILES: CCc1cc2ccc(CN3C[C@@H](Oc4ccc(C(=O)NC)nc4F)[C@@H]3C)c(F)c2[nH]c1=O]

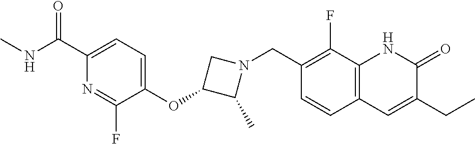 [SMILES: CCc1cc2ccc(CN3C[C@@H](Oc4ccc(C(=O)NC)nc4F)[C@H]3C)c(F)c2[nH]c1=O]

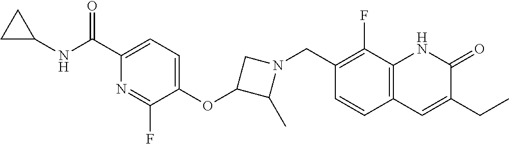 [SMILES: CCc1cc2ccc(CN3CC(Oc4ccc(C(=O)NC5CC5)nc4F)C3C)c(F)c2[nH]c1=O]

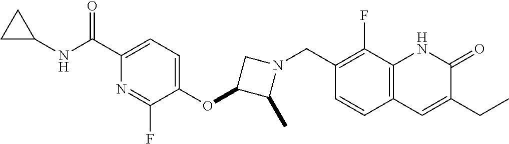 [SMILES: CCc1cc2ccc(CN3C[C@H](Oc4ccc(C(=O)NC5CC5)nc4F)[C@@H]3C)c(F)c2[nH]c1=O]

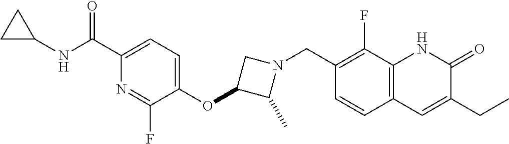 [SMILES: CCc1cc2ccc(CN3C[C@H](Oc4ccc(C(=O)NC5CC5)nc4F)[C@H]3C)c(F)c2[nH]c1=O]

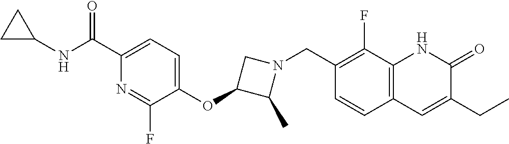 [SMILES: CCc1cc2ccc(CN3C[C@H](Oc4ccc(C(=O)NC5CC5)nc4F)[C@@H]3C)c(F)c2[nH]c1=O]

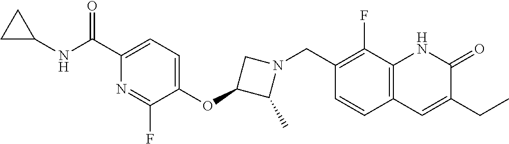 [SMILES: CCc1cc2ccc(CN3C[C@H](Oc4ccc(C(=O)NC5CC5)nc4F)[C@H]3C)c(F)c2[nH]c1=O]

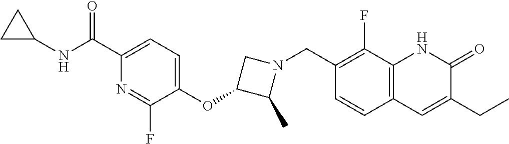 [SMILES: CCc1cc2ccc(CN3C[C@@H](Oc4ccc(C(=O)NC5CC5)nc4F)[C@@H]3C)c(F)c2[nH]c1=O]

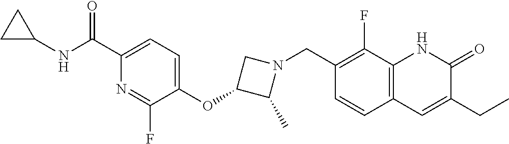 [SMILES: CCc1cc2ccc(CN3C[C@@H](Oc4ccc(C(=O)NC5CC5)nc4F)[C@H]3C)c(F)c2[nH]c1=O]

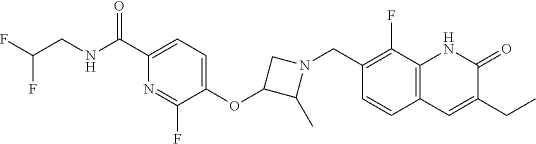 [SMILES: CCc1cc2ccc(CN3CC(Oc4ccc(C(=O)NCC(F)F)nc4F)C3C)c(F)c2[nH]c1=O]

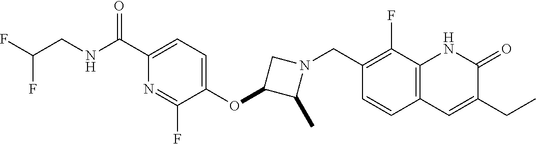 [SMILES: CCc1cc2ccc(CN3C[C@H](Oc4ccc(C(=O)NCC(F)F)nc4F)[C@@H]3C)c(F)c2[nH]c1=O]

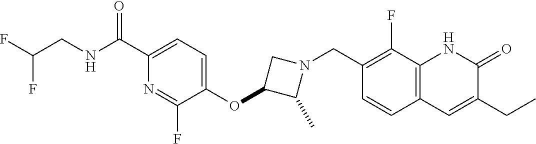 [SMILES: CCc1cc2ccc(CN3C[C@H](Oc4ccc(C(=O)NCC(F)F)nc4F)[C@H]3C)c(F)c2[nH]c1=O]

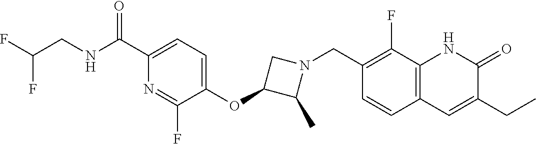 [SMILES: CCc1cc2ccc(CN3C[C@H](Oc4ccc(C(=O)NCC(F)F)nc4F)[C@@H]3C)c(F)c2[nH]c1=O]

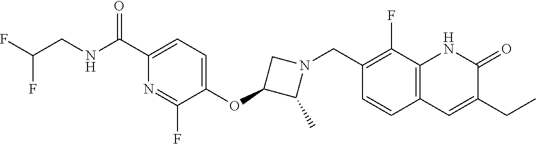 [SMILES: CCc1cc2ccc(CN3C[C@H](Oc4ccc(C(=O)NCC(F)F)nc4F)[C@H]3C)c(F)c2[nH]c1=O]

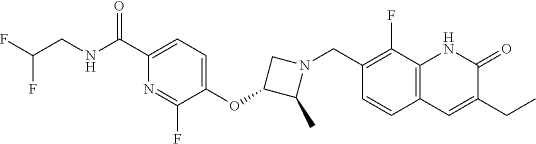 [SMILES: CCc1cc2ccc(CN3C[C@@H](Oc4ccc(C(=O)NCC(F)F)nc4F)[C@@H]3C)c(F)c2[nH]c1=O]

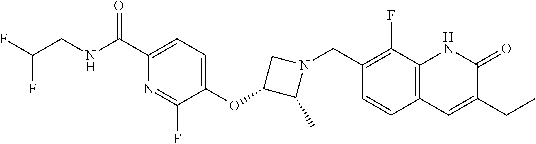 [SMILES: CCc1cc2ccc(CN3C[C@@H](Oc4ccc(C(=O)NCC(F)F)nc4F)[C@H]3C)c(F)c2[nH]c1=O]

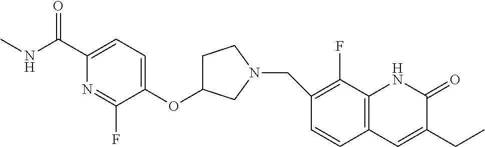 [SMILES: CCc1cc2ccc(CN3CCC(Oc4ccc(C(=O)NC)nc4F)C3)c(F)c2[nH]c1=O]

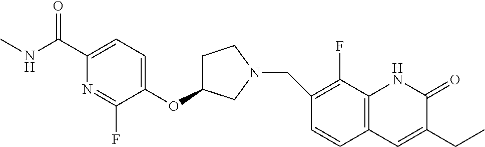 [SMILES: CCc1cc2ccc(CN3CC[C@H](Oc4ccc(C(=O)NC)nc4F)C3)c(F)c2[nH]c1=O]

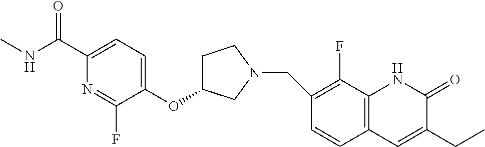 [SMILES: CCc1cc2ccc(CN3CC[C@@H](Oc4ccc(C(=O)NC)nc4F)C3)c(F)c2[nH]c1=O]

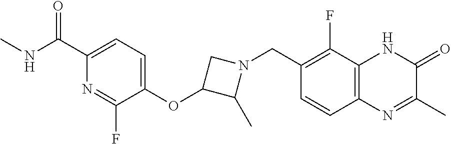

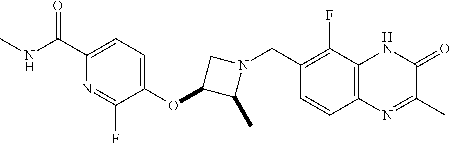

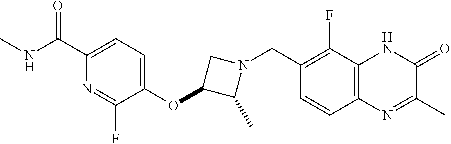

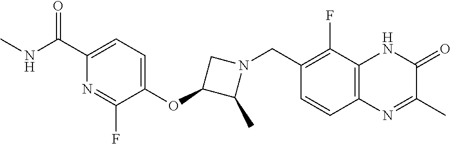

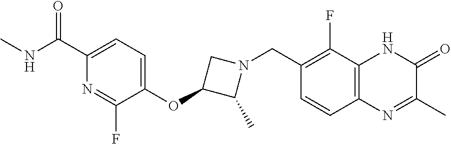

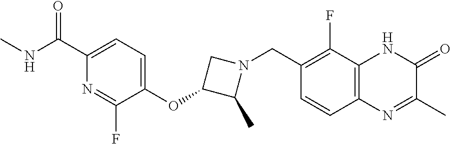

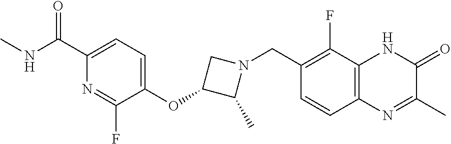

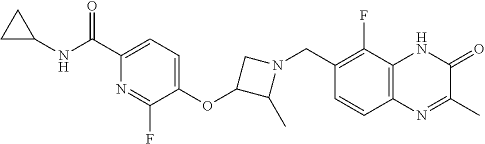 [SMILES: Cc1nc2ccc(CN3CC(Oc4ccc(C(=O)NC5CC5)nc4F)C3C)c(F)c2[nH]c1=O]

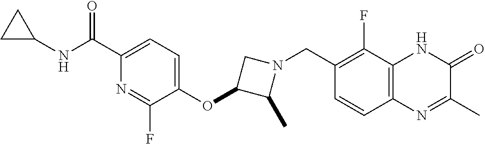 [SMILES: Cc1nc2ccc(CN3C[C@H](Oc4ccc(C(=O)NC5CC5)nc4F)[C@@H]3C)c(F)c2[nH]c1=O]

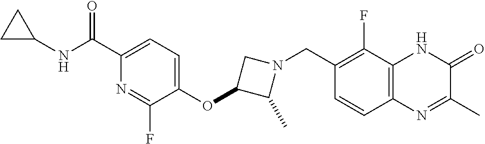 [SMILES: Cc1nc2ccc(CN3C[C@H](Oc4ccc(C(=O)NC5CC5)nc4F)[C@H]3C)c(F)c2[nH]c1=O]

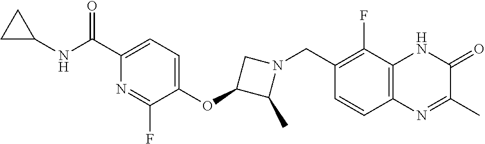 [SMILES: Cc1nc2ccc(CN3C[C@H](Oc4ccc(C(=O)NC5CC5)nc4F)[C@@H]3C)c(F)c2[nH]c1=O]

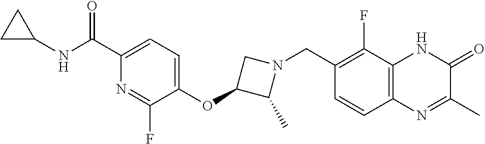 [SMILES: Cc1nc2ccc(CN3C[C@H](Oc4ccc(C(=O)NC5CC5)nc4F)[C@H]3C)c(F)c2[nH]c1=O]

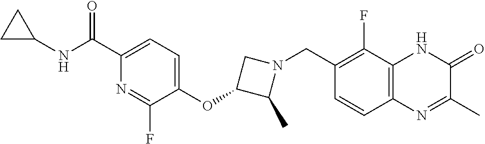 [SMILES: Cc1nc2ccc(CN3C[C@@H](Oc4ccc(C(=O)NC5CC5)nc4F)[C@@H]3C)c(F)c2[nH]c1=O]

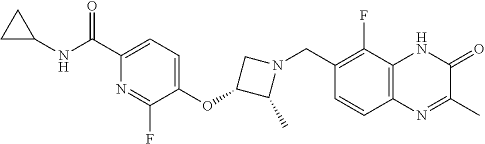 [SMILES: Cc1nc2ccc(CN3C[C@@H](Oc4ccc(C(=O)NC5CC5)nc4F)[C@H]3C)c(F)c2[nH]c1=O]

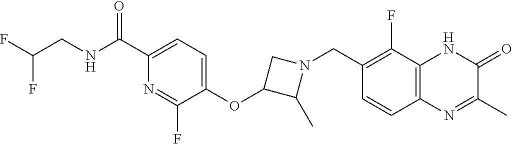 [SMILES: Cc1nc2ccc(CN3CC(Oc4ccc(C(=O)NCC(F)F)nc4F)C3C)c(F)c2[nH]c1=O]

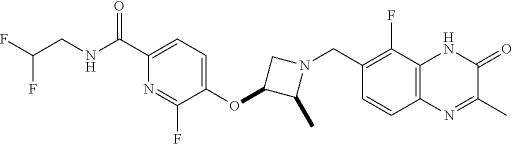 [SMILES: Cc1nc2ccc(CN3C[C@H](Oc4ccc(C(=O)NCC(F)F)nc4F)[C@@H]3C)c(F)c2[nH]c1=O]

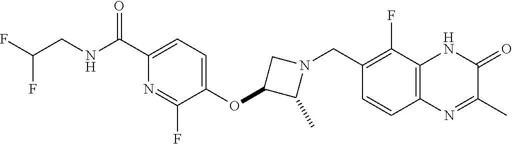 [SMILES: Cc1nc2ccc(CN3C[C@H](Oc4ccc(C(=O)NCC(F)F)nc4F)[C@H]3C)c(F)c2[nH]c1=O]

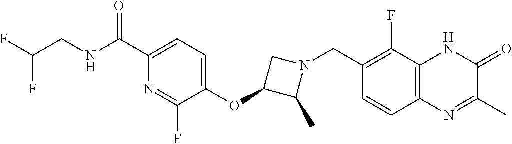 [SMILES: Cc1nc2ccc(CN3C[C@H](Oc4ccc(C(=O)NCC(F)F)nc4F)[C@@H]3C)c(F)c2[nH]c1=O]

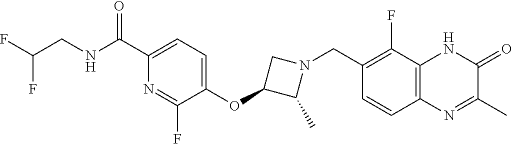 [SMILES: Cc1nc2ccc(CN3C[C@H](Oc4ccc(C(=O)NCC(F)F)nc4F)[C@H]3C)c(F)c2[nH]c1=O]

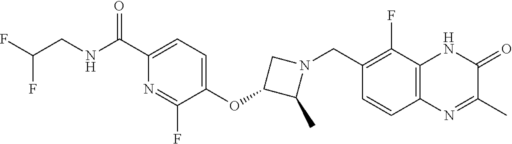 [SMILES: Cc1nc2ccc(CN3C[C@@H](Oc4ccc(C(=O)NCC(F)F)nc4F)[C@@H]3C)c(F)c2[nH]c1=O]

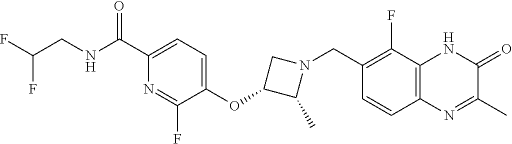 [SMILES: Cc1nc2ccc(CN3C[C@@H](Oc4ccc(C(=O)NCC(F)F)nc4F)[C@H]3C)c(F)c2[nH]c1=O]

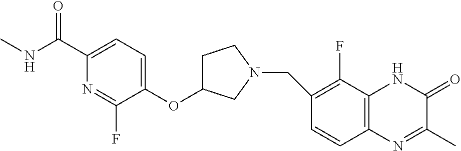

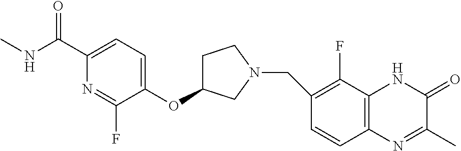

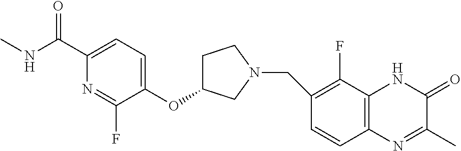

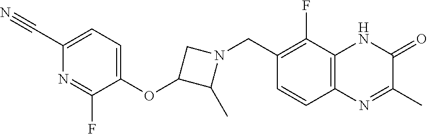 [SMILES: Cc1nc2ccc(CN3CC(Oc4ccc(C#N)nc4F)C3C)c(F)c2[nH]c1=O]

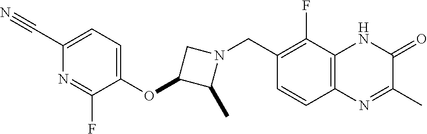 [SMILES: Cc1nc2ccc(CN3C[C@H](Oc4ccc(C#N)nc4F)[C@@H]3C)c(F)c2[nH]c1=O]

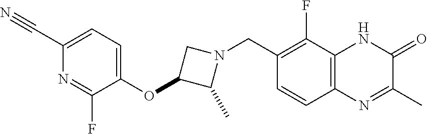 [SMILES: Cc1nc2ccc(CN3C[C@H](Oc4ccc(C#N)nc4F)[C@H]3C)c(F)c2[nH]c1=O]

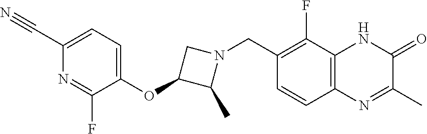 [SMILES: Cc1nc2ccc(CN3C[C@H](Oc4ccc(C#N)nc4F)[C@@H]3C)c(F)c2[nH]c1=O]

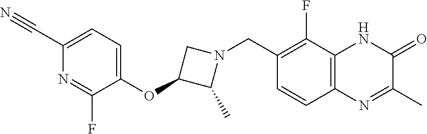 [SMILES: Cc1nc2ccc(CN3C[C@H](Oc4ccc(C#N)nc4F)[C@H]3C)c(F)c2[nH]c1=O]

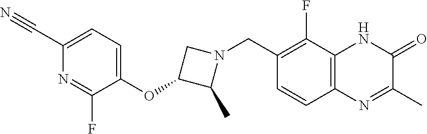 [SMILES: Cc1nc2ccc(CN3C[C@@H](Oc4ccc(C#N)nc4F)[C@@H]3C)c(F)c2[nH]c1=O]

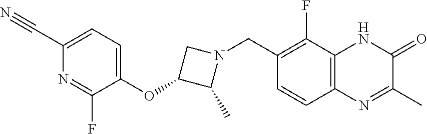 [SMILES: Cc1nc2ccc(CN3C[C@@H](Oc4ccc(C#N)nc4F)[C@H]3C)c(F)c2[nH]c1=O]

### Further Forms and Preparation of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid (wherein “aryl” refers to a radical derived from a hydrocarbon ring system comprising 6 to 30 carbon atoms and at least one aromatic ring, and the aryl radical may be a monocyclic, bicyclic, tricyclic, or tetracyclic ring system), methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof, and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl) 4 (wherein “alkyl” refers to a straight-chain or branched-chain saturated hydrocarbon monoradical having from one to about ten carbon atoms, such as one to six carbon atoms), and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Syntheses

The compounds described herein may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

It will be appreciated that where typical process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups (“PG”) may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein. For example, protecting groups for alcohols, such as hydroxy, include silyl ethers (including trimethylsilyl (TMS), tert-butyldimethylsilyl (TBDMS), tri-iso-propylsilyloxymethyl (TOM), and triisopropylsilyl (TIPS) ethers), which can be removed by acid or fluoride ion, such as NaF, TBAF (tetra-n-butylammonium fluoride), HF-Py, or HF-NEt₃. Other protecting groups for alcohols include acetyl, removed by acid or base, benzoyl, removed by acid or base, benzyl, removed by hydrogenation, methoxyethoxymethyl ether, removed by acid, dimethoxytrityl, removed by acid, methoxymethyl ether, removed by acid, tetrahydropyranyl or tetrahydrofuranyl, removed by acid, and trityl, removed by acid. Examples of protecting groups for amines include carbobenzyloxy, removed by hydrogenolysis, p-methoxybenzyl carbonyl, removed by hydrogenolysis, tert-butyloxycarbonyl, removed by concentrated strong acid (such as HCl or CF₃COOH), or by heating to greater than about 80° C., 9-fluorenylmethyloxycarbonyl, removed by base, such as piperidine, acetyl, removed by treatment with a base, benzoyl, removed by treatment with a base, benzyl, removed by hydrogenolysis, carbamate group, removed by acid and mild heating, p-methoxybenzyl, removed by hydrogenolysis, 3,4-dimethoxybenzyl, removed by hydrogenolysis, p-methoxyphenyl, removed by ammonium cerium (IV) nitrate, tosyl, removed by concentrated acid (such as HBr or H₂SO₄) and strong reducing agents (sodium in liquid ammonia or sodium naphthalenide), troc (trichloroethyl chloroformate), removed by Zn insertion in the presence of acetic acid, and sulfonamides (Nosyl & Nps), removed by samarium iodide or tributyltin hydride.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Method of Treatment

Some embodiments provide for a method of treating a disease or disorder associated with PARP comprising administering a compound disclosed herein, or a pharmaceutically acceptable salt, solvate, or stereoisomer thereof. Some embodiments provide for a method of treating a disease or disorder associated with PARP1 comprising administering a compound disclosed herein, or a pharmaceutically acceptable salt, solvate, or stereoisomer thereof.

Disclosed herein are methods of treatment of a disease in which inhibition of PARP is beneficial, the method comprising administering a compound disclosed herein. Also disclosed herein are methods of treatment of a disease in which inhibition of PARP1 is beneficial, the method comprising administering a compound disclosed herein. In some embodiments, the disease is cancer.

Some embodiments provide for a method of treating cancer in a subject in need thereof, the method comprising administering a therapeutically effective amount of a compound disclosed herein, or a pharmaceutically acceptable salt thereof.

In some embodiments, the cancer is breast cancer, ovarian cancer, pancreatic cancer, prostate cancer, a hematological cancer, a gastrointestinal cancer such as gastric cancer and colorectal cancer, or lung cancer. In some embodiments, the cancer is breast cancer, ovarian cancer, pancreatic cancer, or prostate cancer. In some embodiment, the cancer is leukemia, colon cancer, glioblastoma, lymphoma, melanoma, or cervical cancer. In some embodiments, the cancer is bladder cancer, brain & CNS cancer, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

In some embodiments, the cancer is metastatic cancer. In some embodiments, the cancer has metastasized in the brain.

In some embodiments, the cancer comprises a BRCA1 and/or a BRCA2 mutation.

In some embodiments, the cancer comprising a BRCA1 and/or a BRCA2 mutation is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

In some embodiments, the cancer is a cancer deficient in Flomologous Recombination (FIR) dependent DNA DSB repair activity. The FIR dependent DNA DSB repair pathway repairs double-strand breaks (DSBs) in DNA via homologous mechanisms to reform a continuous DNA helix. The components of the FIR dependent DNA DSB repair pathway include, but are not limited to, ATM (NM_000051), RAD51 (NM_002875), RAD51 L1 (NM_002877), RAD51 C (NM_002876), RAD51 L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE1 1 A (NM_005590) and NBS1 (NM_002485). Other proteins involved in the FIR dependent DNA DSB repair pathway include regulatory factors such as EMSY. In some embodiments, the cancer which is deficient in FIR dependent DNA DSB repair comprises one or more cancer cells which have a reduced or abrogated ability to repair DNA DSBs through that pathway, relative to normal cells i.e., the activity of the FIR dependent DNA DSB repair pathway may be reduced or abolished in the one or more cancer cells.

In some embodiments, the activity of one or more components of the FIR dependent DNA DSB repair pathway is abolished in the one or more cancer cells of an individual having a cancer which is deficient in FIR dependent DNA DSB repair.

In some embodiments, the cancer cells have a BRCA1 and/or a BRCA2 deficient phenotype i.e., BRCA1 and/or BRCA2 activity is reduced or abolished in the cancer cells. Cancer cells with this phenotype may be deficient in BRCA1 and/or BRCA2, i.e., expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor. BRCA1 and BRCA2 are known tumor suppressors whose wild-type alleles are frequently lost in tumors of heterozygous carriers. Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast cancer, ovarian cancer, pancreatic cancer, prostate cancer, a hematological cancer, gastrointestinal cancer, and lung cancer.

Also disclosed herein is a method of treating a cancer comprising a mutation in a gene conferring homologous repair deficiency in a subject in need thereof, the method comprising administering a compound disclosed herein, or a pharmaceutically acceptable salt, solvate, or stereoisomer thereof. In some embodiments, the mutation in a gene conferring homologous repair deficiency comprises ATM, BRCA1, BRCA2, BARD1, BRIP1, CDK12, CHEK1, CHEK2, FANCL, PALB2, RAD51B, RAD51C, RAD51D, or RAD54L, or any combinations thereof.

Also disclosed herein is a method for treating a cancer that is present in the brain, the method comprising administering a compound disclosed herein, or a pharmaceutically acceptable salt, solvate, or stereoisomer thereof.

In some embodiments, the cancer that is present in the brain arises from primary peripheral tumors that have metastasized to the brain. In some embodiments, the cancer that is present in the brain arises from primary brain tissues.

Also disclosed herein is a method for treating brain cancer, the method comprising administering a compound disclosed herein, or a pharmaceutically acceptable salt, solvate, or stereoisomer thereof. Some embodiments provide for a method of treating brain cancer in a subject in need thereof, the method comprising administering a therapeutically effective amount of a compound disclosed herein, or a pharmaceutically acceptable salt thereof

In some embodiments, the brain cancer is a primary brain tumor that starts in the brain and tends to stay there.

In some embodiments, the brain cancer is a secondary brain tumor. These cancers start somewhere else in the body and travel to the brain. Lung, breast, kidney, colon, and skin cancers are among the most common cancers that spread to the brain.

In some embodiments, the compound disclosed herein, or a pharmaceutically acceptable salt, solvate, or stereoisomer thereof, is capable of penetrating the blood brain barrier (BBB). In some embodiments, the ratio of compound that penetrates the BBB is >0.1, wherein 1 is complete BBB penetration, and 0 is no penetration. In some embodiments, the ratio of compound that penetrates the BBB is >0.2. In some embodiments, the ratio of compound that penetrates the BBB is >0.3. In some embodiments, the ratio of compound that penetrates the BBB is measured using the rat kp, uu assay. In some embodiments, the compound has a ratio of >0.3 (i.e. from 0.3 to 1) as determined in the rat kp,uu assay.

### Dosing

In certain embodiments, the compositions containing the compound(s) described herein (or pharmaceutically acceptable salts, solvates, or stereoisomers thereof) are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, to prevent a return of the symptoms of the disease or condition.

In certain embodiments wherein the patient's condition does not improve, upon the doctor's discretion, the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease, condition, and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein, or a pharmaceutically acceptable salt, solvate, or stereoisomer thereof, is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

### Pharmaceutical Compositions/Formulations

The compounds described herein, or a pharmaceutically acceptable salts, solvates, or stereoisomers thereof, are administered to a subject in need thereof, either alone or in combination with pharmaceutically acceptable carriers, excipients, or diluents, in a pharmaceutical composition, according to standard pharmaceutical practice. In one embodiment, the compounds of this disclosure may be administered to animals. The compounds can be administered orally or parenterally, including the intravenous, intramuscular, intraperitoneal, subcutaneous, rectal, and topical routes of administration.

In another aspect, provided herein are pharmaceutical compositions comprising a compound described herein, or a pharmaceutically acceptable salt, solvate, or stereoisomer thereof, and at least one pharmaceutically acceptable excipient. Some embodiments provide for pharmaceutical compositions comprising a compound described herein, or a pharmaceutically acceptable salt thereof, and a pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof, are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are methods of treating cancer using a compound disclosed herein, or a pharmaceutically acceptable salt, solvate, or stereoisomer thereof, in combination with an additional therapeutic agent.

In some embodiments, the additional therapeutic agent is an anticancer agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

### EXAMPLES

The following examples are included to demonstrate specific embodiments of the disclosure. It should be appreciated by those skilled in the art that the techniques disclosed in the examples which follow represent techniques to function well in the practice of the disclosure, and thus can be considered to constitute specific modes of its practice. However, those of skill in the art should, in light of the present disclosure, appreciate that many changes can be made in the specific embodiments which are disclosed and still obtain a like or similar result without departing from the spirit and scope of the disclosure.

### List of Abbreviations and Acronyms

| Abbreviation | Meaning |
|---|---|
| ACN/MeCN/CH₃CN | acetonitrile |
| aq. | aqueous |
| BTC | bis(trichloromethyl)carbonate |
| Boc₂O | di-tert-butyl dicarbonate |
| CH₃NH₂ | methylamine |
| CH₃NH₂HCl | methylamine hydrochloride |
| conc. | concentrated |
| DCM/CH₂Cl₂ | dichloromethane |
| DDQ | 2,3-dichloro-5,6-dicyano-1,4-benzoquinone |
| DEAD | diethyl azodicarboxylate |
| DIAD | diisopropyl azodicarboxylate |
| DIEA | N,N-diisopropylethylamine |
| DMA | dimethylacetamide |
| DMAP | 4-dimethylaminopyridine |
| DMF | N,N-dimethylformamide |
| DMSO | dimethylsulfoxide |
| dppf | 1,1′-bis(diphenylphosphino)ferrocene |
| EtOAc/EA | ethyl acetate |
| equiv | equivalents |
| FA | formic acid |
| h | hour(s) |
| HATU | 1-[bis(dimethylamino)methylene]-1h-1,2,3- |
| | triazolo[4,5-b]pyridinium 3-oxide |
| | hexafluorophosphate |
| HCl | hydrochloric acid |
| H₂SO₄ | sulfuric acid |
| HPLC | high performance liquid chromatography |
| KI | potassium iodide |
| LCMS/LC-MS | liquid chromatography-mass spectrometry |
| LDA | lithium diisopropylamide |
| LiOH | lithium hydroxide |
| MeOH | methanol |
| Na₂SO₄ | sodium sulfate |
| NH₃H₂O | aqueous ammonia |
| NH₄HCO₃ | ammonium bicarbonate |
| Pd₂(dba)₃-CHCl₃ | tris(dibenzylideneacetone)dipalladium(0)- |
| | chloroform adduct |
| PE | petroleum ether |
| PPh₃ | triphenylphosphine |
| SOCl₂ | thionyl chloride |
| TEA | triethylamine |
| TFA | trifluoroacetic acid |
| THF | tetrahydrofuran |
| TPPO | triphenylphosphine oxide |
| XPhos | dicyclohexyl[2′,4′,6′-tris(propan-2-yl)[1,1′- |
| | biphenyl]-2-yl]phosphane |

### Example 1: Synthesis of 6-fluoro-5-{[(3R)-1-({6-fluoro-4-oxo-2H,3H,5H-furo[3,2-c]quinolin-7-yl}methyl)pyrrolidin-3-yl]oxy}-N-methylpyridine-2-carboxamide (Compound 1)

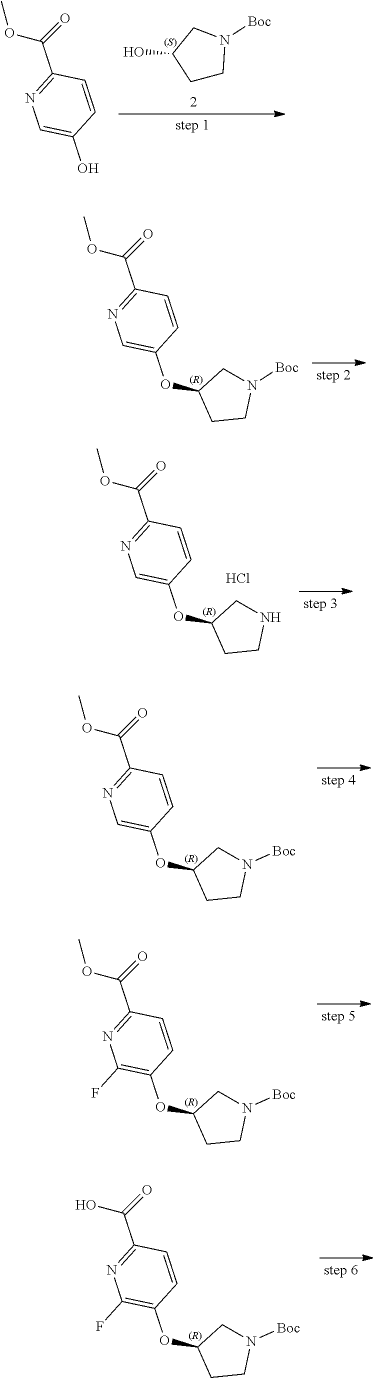

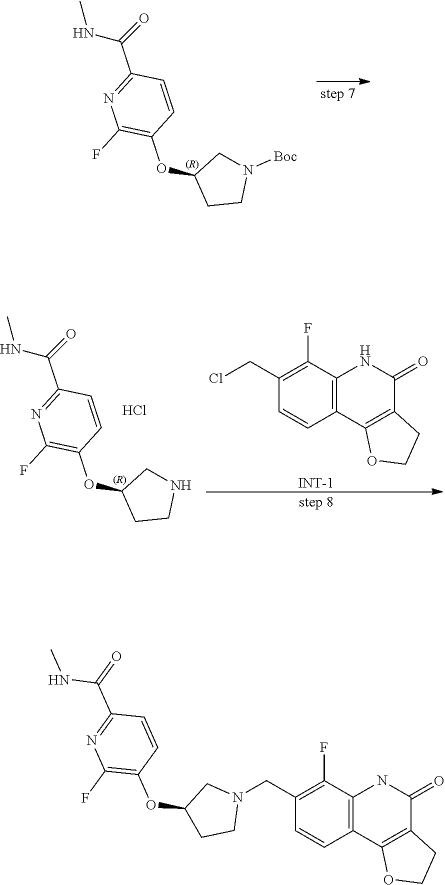

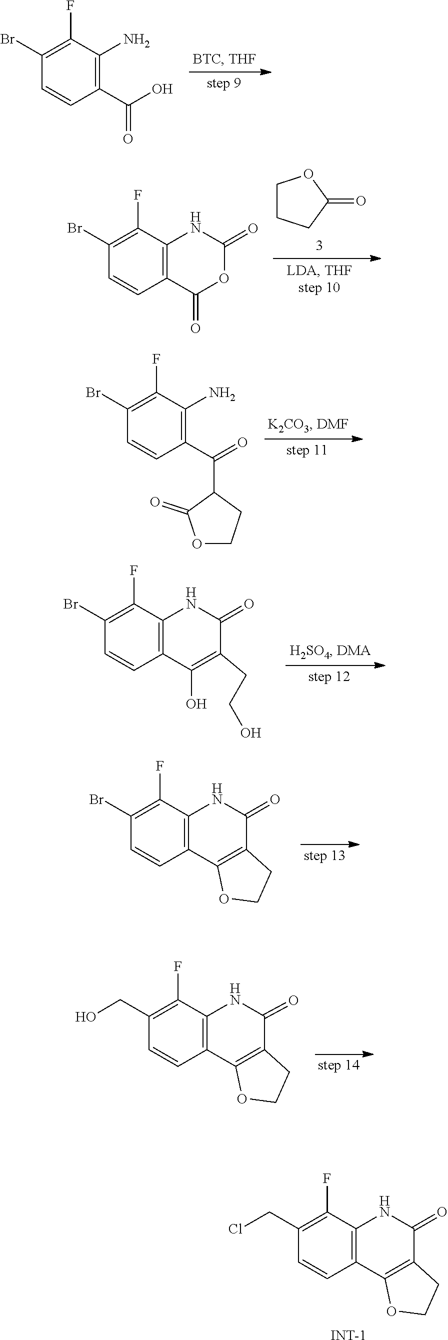

### Step 1: Preparation of methyl 5-{[(3R)-1-(tert-butoxycarbonyl)pyrrolidin-3-yl]oxy}pyridine-2-carboxylate

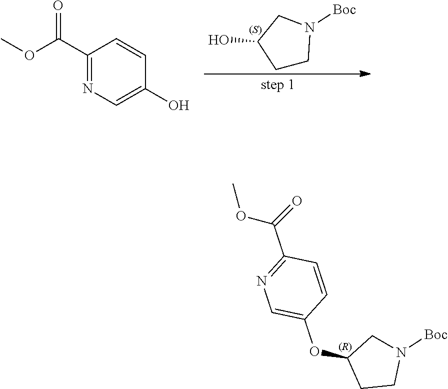

To a stirred solution of methyl 5-hydroxypyridine-2-carboxylate (5.00 g, 6.53 mmol, 1.00 equiv) were added tert-butyl (3S)-3-hydroxypyrrolidine-1-carboxylate (9.17 g, 48.977 mmol, 1.5 equiv), PPh₃ (17.13 g, 65.302 mmol, 2 equiv), and THF (60 mL) at room temperature. To the above mixture was added DEAD (11.37 g, 65.302 mmol, 2 equiv) dropwise over 3 min at 0° C. The resulting mixture was stirred for additional 1.5 h at room temperature. The resulting mixture was extracted with EtOAc (3×50 mL). The combined organic layers were washed with brine (3×50 mL), and dried over anhydrous Na₂SO₄. After filtration, the filtrate was concentrated under reduced pressure. The residue was purified by silica gel column chromatography, and eluted with PE/EA (1:1), to afford methyl 5-{[(3R)-1-(tert-butoxycarbonyl)pyrrolidin-3-yl]oxy}pyridine-2-carboxylate (8 g, 76.01%, contained TPPO).

LC-MS: (ES+H, m/z): [M+H]⁺=323.

### Step 2: Preparation of methyl 5-[(3R)-pyrrolidin-3-yloxy]pyridine-2-carboxylate, HCl salt

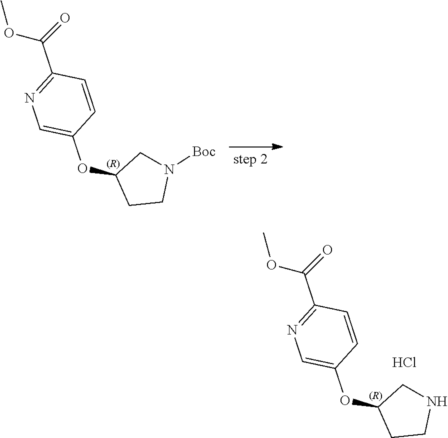

Into a 500 mL vial were added methyl 5-{[(3R)-1-(tert-butoxycarbonyl)pyrrolidin-3-yl]oxy}pyridine-2-carboxylate (7 g, 21.715 mmol, 1 equiv) and DCM (70 mL) at room temperature. To the above mixture was added HCl (gas) in 1,4-dioxane (35 mL, 1151.947 mmol, 53.05 equiv) dropwise over 3 min at 0° C. The resulting mixture was stirred for 1 h at room temperature under nitrogen atmosphere. Desired product could be detected by LCMS. The resulting mixture was concentrated under reduced pressure. The crude product/resulting mixture was used in the next step directly without further purification. This resulted in methyl 5-[(3R)-pyrrolidin-3-yloxy]pyridine-2-carboxylate, HCl salt (3 g, 62.16%, little TPPO).

LC-MS: (ES+H, m/z): [M+H]+=223.

### Step 3: Preparation of methyl 5-{[(3R)-1-(tert-butoxycarbonyl)pyrrolidin-3-yl]oxy}pyridine-2-carboxylate

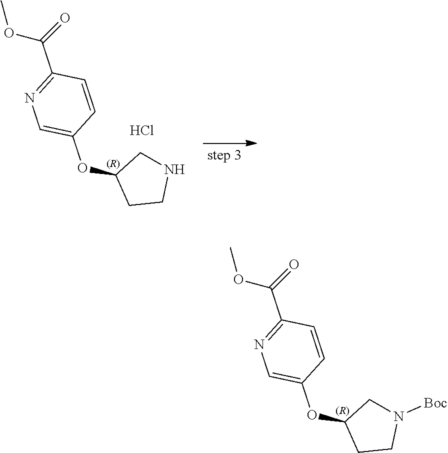

Into a 100 mL vial were added methyl 5-[(3R)-pyrrolidin-3-yloxy]pyridine-2-carboxylate, HCl salt (3 g, 13.499 mmol, 1 equiv), TEA (5.46 g, 53.996 mmol, 4 equiv), DMAP (82.46 mg, 0.675 mmol, 0.05 equiv), and DCM (30 mL) at room temperature. To the above mixture was added Boc₂O (3.54 g, 16.199 mmol, 1.2 equiv) dropwise over 3 min at 0° C. The resulting mixture was stirred for additional 2 h at 40° C. Desired product could be detected by LCMS. To the above mixture was added (2-aminoethyl)dimethylamine (356.98 mg, 4.050 mmol, 0.3 equiv) dropwise over 2 min at 40° C. The resulting mixture was stirred for additional 1 h at 40° C. The resulting mixture was extracted with EtOAc (3×50 mL). The combined organic layers were washed with brine (5×50 mL) and dried over anhydrous Na₂SO₄. After filtration, the filtrate was concentrated under reduced pressure. After filtration, the filtrate was concentrated under reduced pressure. This resulted in methyl 5-{[(3R)-1-(tert-butoxycarbonyl)pyrrolidin-3-yl]oxy}pyridine-2-carboxylate (3.3 g, 75.84%).

LC-MS: (ES+H, m/z): [M+H]⁺=323.

### Step 4: Preparation of methyl 5-{[(3R)-1-(tert-butoxycarbonyl)pyrrolidin-3-yl]oxy}-6-fluoropyridine-2-carboxylate

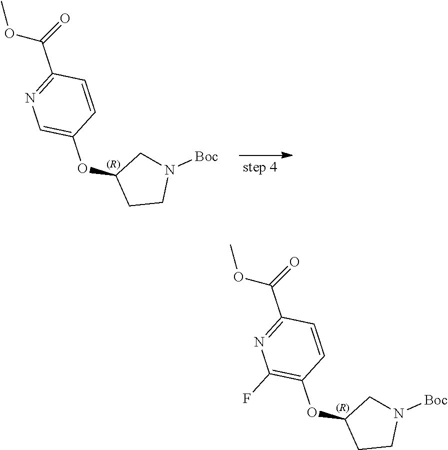

Into a 40 mL vial were added methyl 5-{[(3R)-1-(tert-butoxycarbonyl)pyrrolidin-3-yl]oxy}pyridine-2-carboxylate (3.2 g, 6.204 mmol, 1 equiv), difluorosilver (4.52 g, 31.020 mmol, 5 equiv), and ACN (100 mL) at room temperature. The resulting mixture was stirred for 24 h at 40° C. under nitrogen atmosphere. Desired product could be detected by LCMS. The resulting mixture was filtered, and the filter cake was washed with ACN (3×50 mL). The filtrate was concentrated under reduced pressure. The residue was purified by silica gel column chromatography, eluted with PE/EA (4:1), to afford methyl 5-{[(3R)-1-(tert-butoxycarbonyl)pyrrolidin-3-yl]oxy}-6-fluoropyridine-2-carboxylate (570 mg, 26.99%).

LC-MS: (ES+H, m/z): [M+H]⁺=341.

### Step 5: Preparation of 5-{[(3R)-1-(tert-butoxycarbonyl)pyrrolidin-3-yl]oxy}-6-fluoropyridine-2-carboxylic acid

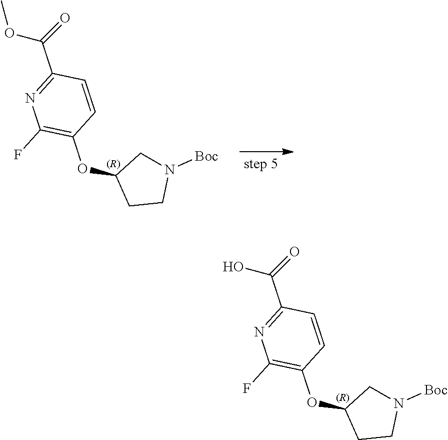

Into a 40 mL vial were added methyl 5-{[(3R)-1-(tert-butoxycarbonyl)pyrrolidin-3-yl]oxy}-6-fluoropyridine-2-carboxylate (500 mg, 1.469 mmol, 1 equiv), LiOH (140.74 mg, 5.876 mmol, 4 equiv), THF (3.5 mL), and H₂O (1.5 mL) at room temperature. The resulting mixture was stirred for 1 h at room temperature under air atmosphere. Desired product could be detected by LCMS. The mixture was acidified to pH 4 with conc. HCl. The resulting mixture was extracted with EtOAc (3×20 mL) and dried over anhydrous Na₂SO₄. After filtration, the filtrate was concentrated under reduced pressure. The crude product was used in the next step directly without further purification. This resulted in 5-{[(3R)-1-(tert-butoxycarbonyl)pyrrolidin-3-yl]oxy}-6-fluoropyridine-2-carboxylic acid (450 mg, 93.87%).

LC-MS: (ES+H, m/z): [M+H]⁺=327.

### Step 6: Preparation of tert-butyl (3R)-3-{[2-fluoro-6-(methylcarbamoyl)pyridin-3-yl]oxy}pyrrolidine-1-carboxylate

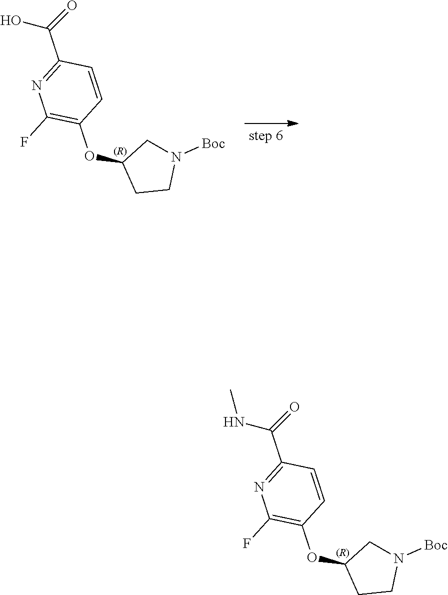

Into a 40 mL vial were added 5-{[(3R)-1-(tert-butoxycarbonyl)pyrrolidin-3-yl]oxy}-6-fluoropyridine-2-carboxylic acid (450 mg, 1.379 mmol, 1 equiv), HATU (786.51 mg, 2.069 mmol, 1.5 equiv), and DMF (4.5 mL) at room temperature. The resulting mixture was stirred for 0.5 h at room temperature under air atmosphere. To the above mixture was added DIEA (712.93 mg, 5.516 mmol, 4 equiv), and CH₃NH₂HCl (85.66 mg, 2.758 mmol, 2 equiv) in portions over 1 min at room temperature. The resulting mixture was stirred for additional 1 h at room temperature. Desired product could be detected by LCMS. The resulting mixture was extracted with EtOAc (3×40 mL). The combined organic layers were washed with brine (3×40 mL) and dried over anhydrous Na₂SO₄. After filtration, the filtrate was concentrated under reduced pressure. The residue was purified by silica gel column chromatography, eluted with PE/EA (5:1), to afford tert-butyl (3R)-3-{[2-fluoro-6-(methylcarbamoyl)pyridin-3-yl]oxy}pyrrolidine-1-carboxylate (400 mg, 85.47%).

LC-MS: (ES+H, m/z): [M+H]⁺=340.

### Step 7: Preparation of 6-fluoro-N-methyl-5-[(3R)-pyrrolidin-3-yloxy]pyridine-2-carboxamide hydrochloride, HCl salt

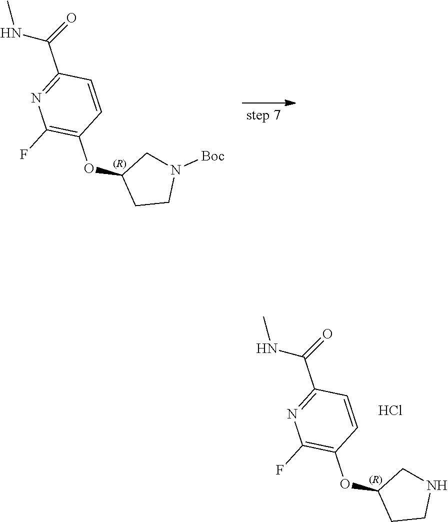

Into a 40 mL vial were added tert-butyl (3R)-3-{[2-fluoro-6-(methylcarbamoyl)pyridin-3-yl]oxy}pyrrolidine-1-carboxylate (400 mg, 1.473 mmol, 1 equiv) and DCM (4 mL) at room temperature. To the above mixture was added HCl (gas) in 1,4-dioxane (2 mL, 82.282 mmol, 55.85 equiv) dropwise over 3 min at 0° C. The resulting mixture was stirred for additional 1 h at room temperature. Desired product could be detected by LCMS. The resulting mixture was concentrated under reduced pressure. The crude product was used in the next step directly without further purification. This resulted in 6-fluoro-N-methyl-5-[(3R)-pyrrolidin-3-yloxy]pyridine-2-carboxamide hydrochloride, HCl salt (400 mg, crude).

LC-MS: (ES+H, m/z): [M+H]⁺=240.

### Step 8: Preparation of 6-fluoro-5-{[(3R)-1-({6-fluoro-4-oxo-2H,3H,5H-furo[3,2-c]quinolin-7-yl}methyl)pyrrolidin-3-yl]oxy}-N-methylpyridine-2-carboxamide

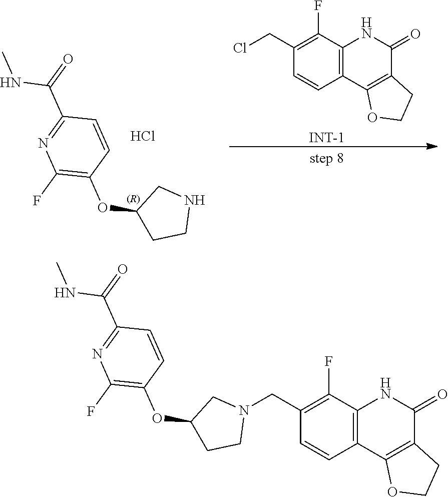

Into a 40 mL vial were added 6-fluoro-N-methyl-5-[(3R)-pyrrolidin-3-yloxy]pyridine-2-carboxamide hydrochloride, HCl salt (400 mg, 1.813 mmol, 1 equiv), 7-(chloromethyl)-6-fluoro-2H,3H,5H-furo[3,2-c]quinolin-4-one (441.6 mg, 2.176 mmol, 1.2 equiv), KI (48.16 mg, 0.363 mmol, 0.2 equiv), DIEA (936 mg, 9.065 mmol, 5 equiv), and ACN (5 mL) at room temperature. The resulting mixture was stirred for 2 h at 80° C. under nitrogen atmosphere. Desired product could be detected by LCMS. The precipitated solids were collected by filtration and washed with ACN (3×20 mL). The crude product (300 mg) was purified by Prep-HPLC with the following conditions (Column: YMC-Actus Triart C18 EXRS 30*150 mm, 5 um; Mobile Phase A: Water (10 mmol/L NH₄HCO₃+0.05% NH₃H₂O), Mobile Phase B: MeOH; Flow rate: 100 mL/min; Gradient: 45% B to 75% B in 30 min; Wave Length: 254 nm/220 nm; RT1 (min): 9.88) to afford 6-fluoro-5-{[(3R)-1-({6-fluoro-4-oxo-2H,3H,5H-furo[3,2-c]quinolin-7-yl}methyl)pyrrolidin-3-yl]oxy}-N-methylpyridine-2-carboxamide (101 mg, 15.20%).

LC-MS: (ES+H, m/z): [M+H]⁺=457.

¹H NMR (400 MHZ, DMSO-d₆) δ 11.39 (s, 1H), 8.44 (q, J=4.7 Hz, 1H), 7.87 (d, J=8.2 Hz, 1H), 7.69 (dd, J=10.1, 8.2 Hz, 1H), 7.39 (d, J=8.2 Hz, 1H), 7.21 (dd, J=8.2, 6.1 Hz, 1H), 5.20-4.96 (m, 1H), 4.81 (t, J=9.3 Hz, 2H), 3.95-3.73 (m, 2H), 3.06 (t, J=9.3 Hz, 2H), 2.93 (dd, J=10.7, 6.0 Hz, 1H), 2.76 (d, J=4.7 Hz, 5H), 2.50 (p, J=1.9 Hz, 1H), 2.47 (d, J=8.2 Hz, 1H), 1.82 (dt, J=15.6, 6.3 Hz, 1H), 1.23 (s, OH).

¹⁹F NMR (377 MHz, DMSO-d₆) δ 84.68, −133.29.

### Step 9: Preparation of 7-bromo-8-fluoro-2H-benzo[d][1,3]oxazine-2,4(1H)-dione

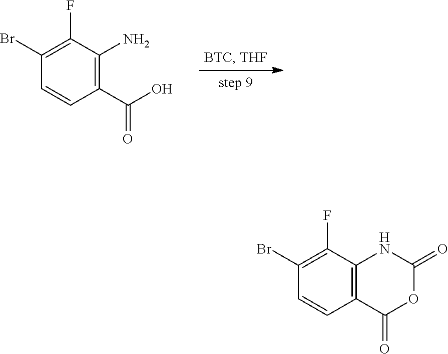 [SMILES: Nc1c(C(=O)O)ccc(Br)c1F ; O=c1[nH]c2c(F)c(Br)ccc2c(=O)o1]

To a stirred mixture of 2-amino-4-bromo-3-fluorobenzoic acid (12 g, 51.72 mmol, 1 equiv.) in THF (200 mL) was added bis(trichloromethyl)carbonate (7.61 g, 25.64 mmol, 0.5 equiv.) dropwise at 0° C. under nitrogen atmosphere. The resulting mixture was stirred for 2 h at room temperature under nitrogen atmosphere. The reaction was monitored by LCMS. The resulting mixture was concentrated under reduced pressure. The residue was dissolved in THF (100 mL). The resulting mixture was stirred for 30 min at room temperature under nitrogen atmosphere. The resulting mixture was concentrated under reduced pressure. The residue was dissolved in hexane (100 mL). The resulting mixture was stirred for 30 min at room temperature under nitrogen atmosphere. The precipitated solids were collected by filtration and washed with hexane (3×100 mL). The resulting mixture was concentrated under reduced pressure to afford 7-bromo-8-fluoro-2H-benzo[d][1,3]oxazine-2,4(1H)-dione (13 g, 97.4%).

m/z [M+H]⁺=260/262.

### Step 10: Preparation of 3-(2-amino-4-bromo-3-fluorobenzoyl)dihydrofuran-2(3H)-one

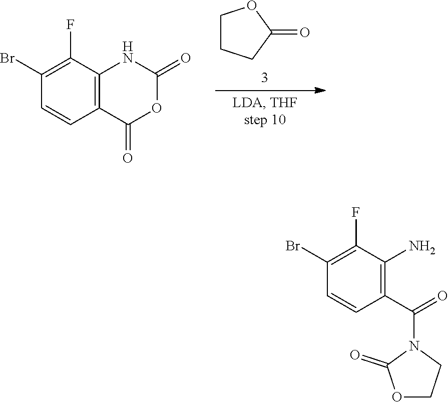

To a stirred solution of 7-bromo-8-fluoro-1H-3,1-benzoxazine-2,4-dione (12 g, 46.15 mmol, 1 equiv.) and 4-butyrolactone (5.96 g, 69.22 mmol, 1.5 equiv.) in THF (150 mL) was added LDA (2M in THF) (14.83 g, 138.45 mmol, 3 equiv.) dropwise at −70° C. under nitrogen atmosphere. The resulting mixture was stirred for 2 h at −70° C. under nitrogen atmosphere. The reaction was monitored by LCMS. Desired product could be detected by LCMS. The reaction was quenched with MeOH (20 mL) at −20° C. The resulting mixture was concentrated under reduced pressure to afford 3-(2-amino-4-bromo-3-fluorobenzoyl)oxolan-2-one (11 g, crude). The crude product was used in the next step directly without further purification.

m/z [M+H]⁺=302/304.

### Step 11: Preparation of 7-bromo-8-fluoro-4-hydroxy-3-(2-hydroxyethyl) quinolin-2 (1H)-one

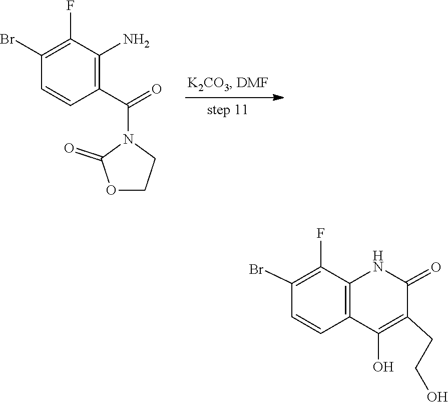 [SMILES: Nc1c(C(=O)N2CCOC2=O)ccc(Br)c1F ; O=c1[nH]c2c(F)c(Br)ccc2c(O)c1CCO]

To the stirred mixture of 3-(2-amino-4-bromo-3-fluorobenzoyl)dihydrofuran-2 (3H)-one (11 g, 36.54 mmol, 1 equiv) was added K₂CO₃ (10.9 g, 79.09 mmol, 2.00 equiv) at room temperature under nitrogen atmosphere. The resulting mixture was stirred for additional 3 h at 80° C. under nitrogen atmosphere. The mixture was allowed to cool down to room temperature. The reaction was monitored by LCMS. The resulting mixture was diluted with ice water (500 mL). The mixture was acidified to pH 4 with citric acid and stirred for 1 h at room temperature. The precipitated solids were collected by filtration and washed with water (3×50 mL). The resulting solid was dried under 45° C. to afford 7-bromo-8-fluoro-4-hydroxy-3-(2-hydroxyethyl) quinolin-2 (1H)-one (10 g, 90.9%).

m/z [M+H]+=302/304.

### Step 12: Preparation of 7-bromo-6-fluoro-3,5-dihydrofuro[3,2-c]quinolin-4(2H)-one

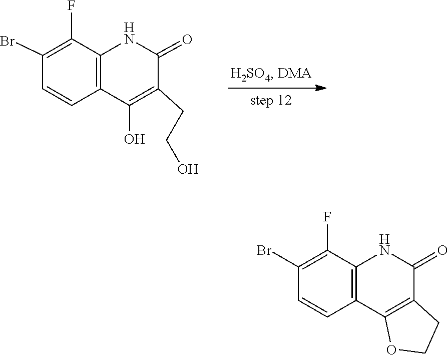 [SMILES: O=c1[nH]c2c(F)c(Br)ccc2c(O)c1CCO ; O=c1[nH]c2c(F)c(Br)ccc2c2c1CCO2]

To a stirred mixture of 7-bromo-8-fluoro-4-hydroxy-3-(2-hydroxyethyl)-1H-quinolin-2-one (10 g, 33.10 mmol, 1 equiv) in DMA (150 mL) was added H₂SO₄ (15 mL, 281.43 mmol, 8.5 equiv) dropwise at 0° C. under nitrogen atmosphere. The resulting mixture was stirred for 3 h at 140° C. under nitrogen atmosphere. The reaction was monitored by LCMS. The reaction was quenched by the addition of water/ice (1000 mL) at 0° C. The precipitated solids were collected by filtration and washed with water (2×100 mL). The residue was purified by trituration with MeOH (50 mL). The precipitated solids were collected by filtration and washed with MeOH (3×10 mL). This resulted in 7-bromo-6-fluoro-3,5-dihydrofuro[3,2-c]quinolin-4(2H)-one (5.6 g, 59.6%).

m/z [M+H]⁺=284/286.

### Step 13: Preparation of 6-fluoro-7-(hydroxymethyl)-3,5-dihydrofuro[3,2-c]quinolin-4(2H)-one

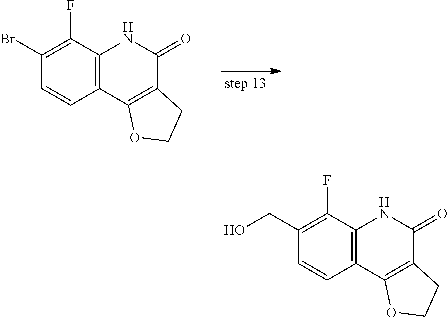 [SMILES: O=c1[nH]c2c(F)c(Br)ccc2c2c1CCO2 ; O=c1[nH]c2c(F)c(CO)ccc2c2c1CCO2]

To a stirred mixture of 7-bromo-6-fluoro-3,5-dihydrofuro[3,2-c]quinolin-4(2H)-one (5.5 g, 19.36 mmol, 1 equiv) and (tributylstannyl) methanol (12.43 g, 38.72 mmol, 2 equiv) in dioxane (80 mL) was added 2nd Generation XPhos precatalyst (1845.94 mg, 3.87 mmol, 0.2 equiv) at room temperature under nitrogen atmosphere. The resulting mixture was stirred overnight at 80° C. under nitrogen atmosphere. The reaction was monitored by LCMS. The resulting mixture was concentrated under reduced pressure. The residue was purified by silica gel column chromatography, eluted with CH₂Cl₂/MeOH (10:1) to afford 6-fluoro-7-(hydroxymethyl)-3,5-dihydrofuro[3,2-c]quinolin-4(2H)-one (3 g, 65.9%).

m/z [M+H]⁺=236.

### Step 14: Preparation of 7-(chloromethyl)-6-fluoro-3,5-dihydrofuro[3,2-c]quinolin-4(2H)-one

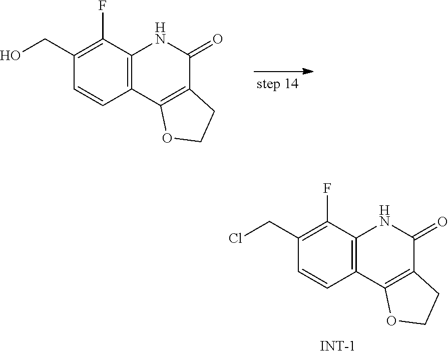 [SMILES: O=c1[nH]c2c(F)c(CO)ccc2c2c1CCO2 ; O=c1[nH]c2c(F)c(CCl)ccc2c2c1CCO2 ; "INT-1"]

To a stirred mixture of 6-fluoro-7-(hydroxymethyl)-3,5-dihydrofuro[3,2-c]quinolin-4(2H)-one (3 g, 12.75 mmol, 1 equiv) and DMF (93 mg, 1.26 mmol, 0.1 equiv) in DCM (60 mL) was added SOCl₂ (5.55 mL, 76.52 mmol, 6 equiv) dropwise at 0° C. under nitrogen atmosphere. The resulting mixture was stirred for 2 h at room temperature under nitrogen atmosphere. The reaction was monitored by LCMS. The resulting mixture was concentrated under reduced pressure. The residue was dissolved in DCM (50 mL). The resulting mixture was concentrated under reduced pressure. This resulted in 7-(chloromethyl)-6-fluoro-3,5-dihydrofuro[3,2-c]quinolin-4(2H)-one (3.2 g, crude).

m/z [M+H]⁺=254.

### Example 2: Synthesis of 6-fluoro-5-{[(2R,3S)-1-({6-fluoro-4-oxo-2H,3H,5H-furo[3,2-c]quinolin-7-yl}methyl)-2-methylazetidin-3-yl]oxy}-N-methylpyridine-2-carboxamide (Compound 2)

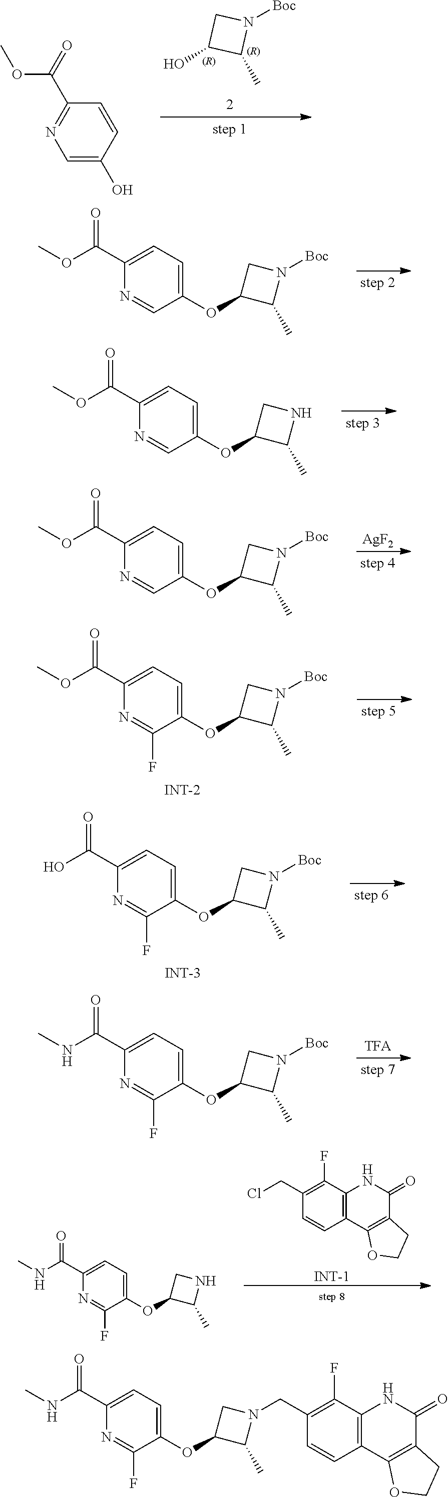

### Step 1: Preparation of methyl 5-{[(2R,3S)-1-(tert-butoxycarbonyl)-2-methylazetidin-3-yl]oxy}pyridine-2-carboxylate

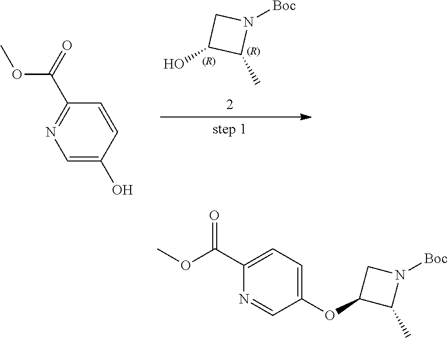

Into a 250 mL round-bottom flask were added methyl 5-hydroxypyridine-2-carboxylate (8 g, 32.651 mmol, 1 equiv), tert-butyl (2R,3R)-3-hydroxy-2-methylazetidine-1-carboxylate (14.67 g, 48.977 mmol, 1.5 equiv), PPh₃ (27.41 g, 65.302 mmol, 2 equiv), and THF (80 mL) at room temperature. To the above mixture was added DEAD (18.19 g, 65.302 mmol, 2 equiv) dropwise over 3 min at 0° C. The resulting mixture was stirred for additional 1.5 h at room temperature. Desired product could be detected by LCMS. The resulting mixture was extracted with EtOAc (3×50 mL). The combined organic layers were washed with brine (3×50 mL), and dried over anhydrous Na₂SO₄. After filtration, the filtrate was concentrated under reduced pressure. The residue was purified by silica gel column chromatography, eluted with PE/EA (1:1), to afford methyl 5-{[(2R,3S)-1-(tert-butoxycarbonyl)-2-methylazetidin-3-yl]oxy}pyridine-2-carboxylate (12 g, 70.71%, contained TPPO).

LC-MS: (ES+H, m/z): [M+H]⁺=323.

### Step 2: Preparation of methyl 5-{[(2R,3S)-2-methylazetidin-3-yl]oxy}pyridine-2-carboxylate, HCl salt

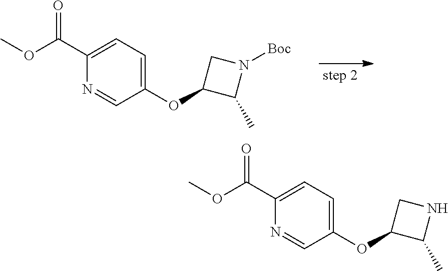

Into a 250 mL vial were added methyl 5-{[(2R,3S)-1-(tert-butoxycarbonyl)-2-methylazetidin-3-yl]oxy}pyridine-2-carboxylate (12 g, 15.511 mmol, 1 equiv) and DCM (120 mL, 786.530 mmol, 50.71 equiv) at room temperature. To the above mixture was added HCl (gas) in 1,4-dioxane (60 mL, 1316.511 mmol, 84.88 equiv) dropwise over 3 min at 0° C. The resulting mixture was stirred for 1 h at room temperature under nitrogen atmosphere. Desired product could be detected by LCMS. The resulting mixture was concentrated under reduced pressure. The crude product was used in the next step directly without further purification. This resulted in methyl 5-{[(2R,3S)-2-methylazetidin-3-yl]oxy}pyridine-2-carboxylate, HCl salt (8 g, little TPPO).

LC-MS: (ES+H, m/z): [M+H]⁺=223.

### Step 3: Preparation of methyl 5-{[(2R,3S)-1-(tert-butoxycarbonyl)-2-methylazetidin-3-yl]oxy}pyridine-2-carboxylate

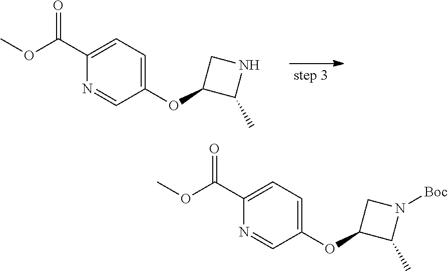

Into a 250 mL vial were added methyl 5-{[(2R,3S)-2-methylazetidin-3-yl]oxy}pyridine-2-carboxylate, HCl salt (8 g, 40.496 mmol, 1 equiv), TEA (14.56 g, 161.984 mmol, 4 equiv), DMAP (219.55 mg, 2.025 mmol, 0.05 equiv), and DCM (80 mL) at room temperature. To the above mixture was added Boc₂O (9.43 g, 48.595 mmol, 1.2 equiv) dropwise over 3 min at 0° C. The resulting mixture was stirred for additional 2 h at 40° C. Desired product could be detected by LCMS. To the above mixture was added (2-aminoethyl)dimethylamine (951.11 mg, 12.149 mmol, 0.3 equiv) dropwise over 3 min at 40° C. The resulting mixture was stirred for additional 1 h at 40° C. The resulting mixture was extracted with EtOAc (3×50 mL) and dried over anhydrous Na₂SO₄. After filtration, the filtrate was concentrated under reduced pressure. The crude product was used in the next step directly without further purification. This resulted in methyl 5-{[(2R,3S)-1-(tert-butoxycarbonyl)-2-methylazetidin-3-yl]oxy}pyridine-2-carboxylate (8.7 g, 66.64%).

LC-MS: (ES+H, m/z): [M+H]⁺=323.

### Step 4: Preparation of methyl 5-{[(2R,3S)-1-(tert-butoxycarbonyl)-2-methylazetidin-3-yl]oxy}-6-fluoropyridine-2-carboxylate

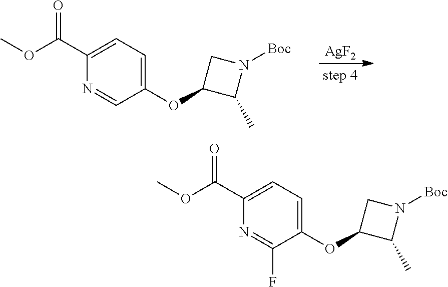

Into a 1000 mL round-bottom flask were added methyl 5-{[(2R,3S)-1-(tert-butoxycarbonyl)-2-methylazetidin-3-yl]oxy}pyridine-2-carboxylate (8 g, 24.817 mmol, 1 equiv), difluorosilver (18.10 g, 124.085 mmol, 5 equiv), and ACN (300 mL) at room temperature. The resulting mixture was stirred for 24 h at 40° C. under nitrogen atmosphere. Desired product could be detected by LCMS. The resulting mixture was filtered, and the filter cake was washed with ACN (3×50 mL). The filtrate was concentrated under reduced pressure. The residue was purified by silica gel column chromatography, eluted with PE/EA (4:1), to afford methyl 5-{[(2R,3S)-1-(tert-butoxycarbonyl)-2-methylazetidin-3-yl]oxy}-6-fluoropyridine-2-carboxylate (1.8 g, 21.31%).

LC-MS: (ES+H, m/z): [M+H]⁺=341.

### Step 5: Preparation of 5-{[(2R,3S)-1-(tert-butoxycarbonyl)-2-methylazetidin-3-yl]oxy}-6-fluoropyridine-2-carboxylic acid

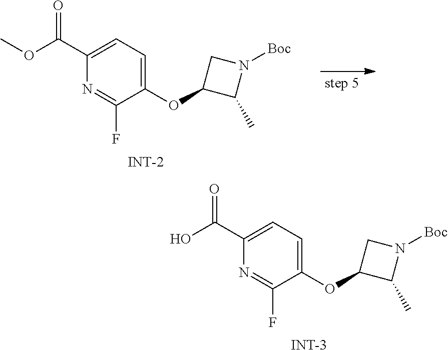

Into a 100 mL vial were added methyl 5-{[(2R,3S)-1-(tert-butoxycarbonyl)-2-methylazetidin-3-yl]oxy}-6-fluoropyridine-2-carboxylate (1.8 g, 6.758 mmol, 1 equiv), LiOH (508.69 mg, 27.032 mmol, 4 equiv), and THF (16 mL), H₂O (4 mL) at room temperature. The resulting mixture was stirred for 1 h at room temperature under nitrogen atmosphere. Desired product could be detected by LCMS. The mixture was acidified to pH 5 with HCl (aq.). The resulting mixture was concentrated under reduced pressure. This resulted in 5-{[(2R,3S)-1-(tert-butoxycarbonyl)-2-methylazetidin-3-yl]oxy}-6-fluoropyridine-2-carboxylic acid (1.75 g, 99.76%).

LC-MS: (ES+H, m/z): [M+H]⁺=327.

### Step 6: Preparation of tert-butyl (2R,3S)-3-{[2-fluoro-6-(methylcarbamoyl)pyridin-3-yl]oxy}-2-methylazetidine-1-carboxylate

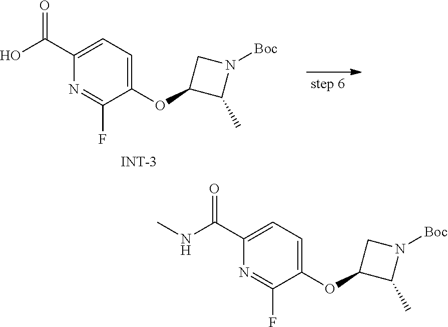

Into a 40 mL vial were added 5-{[(2R,3S)-1-(tert-butoxycarbonyl)-2-methylazetidin-3-yl]oxy}-6-fluoropyridine-2-carboxylic acid (600 mg, 1.763 mmol, 1 equiv), HATU (1.01 g, 2.644 mmol, 1.5 equiv), DIEA (1.14 g, 8.815 mmol, 5 equiv), and DMF (6 mL) at room temperature. The resulting mixture was stirred for 0.5 h at room temperature under nitrogen atmosphere. To the above mixture was added CH₃NH₂ (65.70 mg, 2.116 mmol, 1.2 equiv) in portions over 1 min at room temperature. The resulting mixture was stirred for additional 1 h at room temperature. Desired product could be detected by LCMS. The resulting mixture was extracted with EtOAc (3×20 mL). The combined organic layers were washed with brine (3×20 mL) and dried over anhydrous Na₂SO₄. After filtration, the filtrate was concentrated under reduced pressure. The resulting mixture was concentrated under reduced pressure. This resulted in tert-butyl (2R,3S)-3-{[2-fluoro-6-(methylcarbamoyl)pyridin-3-yl]oxy}-2-methylazetidine-1-carboxylate (590 mg, 98.62%).

LC-MS: (ES+H, m/z): [M+H]⁺=340.

### Step 7: Preparation of 6-fluoro-N-methyl-5-{[(2R,3S)-2-methylazetidin-3-yl]oxy}pyridine-2-carboxamide, TFA salt

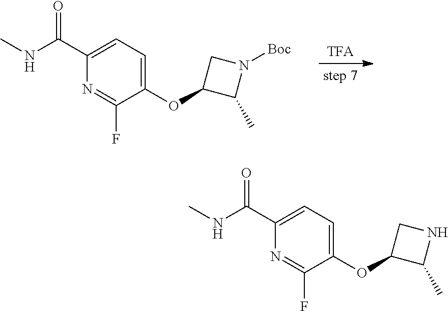

Into a 40 mL vial were added tert-butyl (2R,3S)-3-{[2-fluoro-6-(methylcarbamoyl)pyridin-3-yl]oxy}-2-methylazetidine-1-carboxylate (550 mg, 1.621 mmol, 1 equiv) and DCM (5 mL) at room temperature. To the above mixture was added trifluoroacetic acid (5 mL, 0.005 mmol, 0.17 equiv) dropwise over 3 min at 0° C. The resulting mixture was stirred for 1 h at room temperature under air atmosphere. Desired product could be detected by LCMS. The resulting mixture was concentrated under reduced pressure. The crude product was used in the next step directly without further purification. This resulted in 6-fluoro-N-methyl-5-{[(2R,3S)-2-methylazetidin-3-yl]oxy}pyridine-2-carboxamide, TFA salt (550 mg, crude).

LC-MS: (ES+H, m/z): [M+H]⁺=240.

### Step 8: Preparation of 6-fluoro-5-{[(2R,3S)-1-({6-fluoro-4-oxo-2H,3H,5H-furo[3,2-c]quinolin-7-yl}methyl)-2-methylazetidin-3-yl]oxy}-N-methylpyridine-2-carboxamide

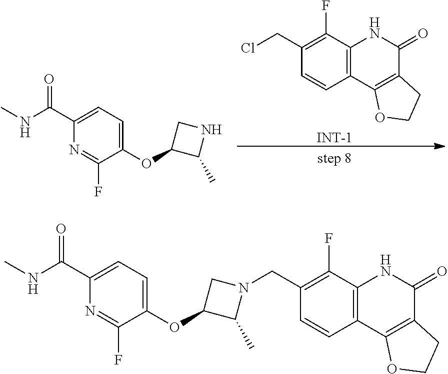

Into a 40 mL vial were added 6-fluoro-N-methyl-5-{[(2R,3S)-2-methylazetidin-3-yl]oxy}pyridine-2-carboxamide, TFA salt (500 mg, 2.090 mmol, 1 equiv), 7-(chloromethyl)-6-fluoro-2H,3H,5H-furo[3,2-c]quinolin-4-one (636.14 mg, 2.508 mmol, 1.2 equiv), KI (69.38 mg, 0.418 mmol, 0.2 equiv), DIEA (1.35 g, 10.450 mmol, 5 equiv), and ACN (5 mL) at room temperature. The resulting mixture was stirred for 2 h at 80° C. under nitrogen atmosphere. Desired product could be detected by LCMS. The precipitated solids were collected by filtration and washed with ACN (3×30 mL). The crude product (400 mg) was purified by Prep-HPLC with the following conditions (Column: YMC-Actus Triart C18 EXRS 30*150 mm, 5 um; Mobile Phase A: Water (0.1% FA), Mobile Phase B: MeOH; Flow rate: 100 mL/min; Gradient: 5% B to 35% B in 30 min; Wave Length: 254 nm/220 nm; RT1 (min): 9.88) to afford 6-fluoro-5-{[(2R,3S)-1-({6-fluoro-4-oxo-2H,3H,5H-furo[3,2-c]quinolin-7-yl}methyl)-2-methylazetidin-3-yl]oxy}-N-methylpyridine-2-carboxamide (101 mg, 10.59%).

LC-MS: (ES+H, m/z): [M+H]⁺=457

¹H NMR (400 MHZ, DMSO-d₆) δ 11.38 (s, 1H), 8.46 (q, J=4.7 Hz, 1H), 7.85 (d, J=8.2 Hz, 1H), 7.60 (dd, J=10.2, 8.2 Hz, 1H), 7.38 (d, J=8.1 Hz, 1H), 7.20 (dd, J=8.1, 6.2 Hz, 1H), 4.81 (t, J=9.3 Hz, 2H), 4.59 (q, J=5.9 Hz, 1H), 3.91 (d, J=13.2 Hz, 1H), 3.81 (t, J=6.4 Hz, 1H), 3.73-3.65 (m, 1H), 3.43-3.35 (m, 1H), 3.06 (t, J=9.3 Hz, 2H), 2.82 (t, J=6.7 Hz, 1H), 2.76 (d, J=4.7 Hz, 3H), 1.19 (d, J=6.1 Hz, 3H).

¹⁹F NMR (377 MHz, DMSO-d₆) δ 84.70, −133.55.

### Example 3: Synthesis of N-cyclopropyl-6-fluoro-5-{[(2R,3S)-1-({6-fluoro-4-oxo-2H,3H,5H-furo[3,2-c]quinolin-7-yl}methyl)-2-methylazetidin-3-yl]oxy}pyridine-2-carboxamide (Compound 3)

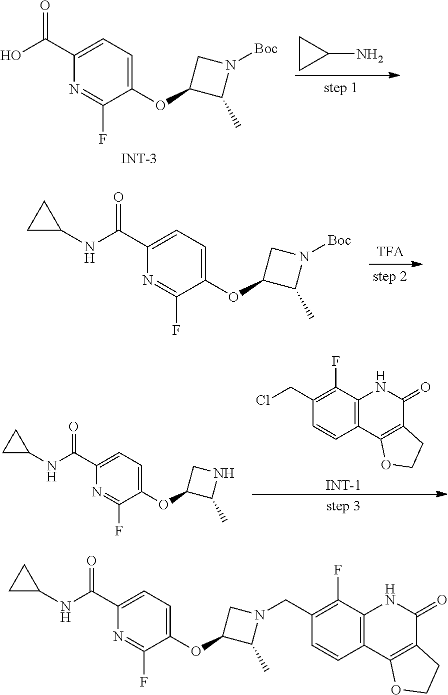

### Step 1: Preparation of tert-butyl (2R,3S)-3-{[6-(cyclopropylcarbamoyl)-2-fluoropyridin-3-yl]oxy}-2-methylazetidine-1-carboxylate

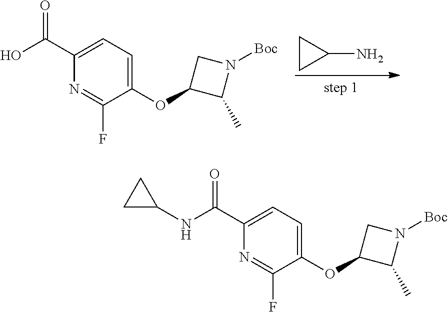

Into a 40 mL vial were added 5-{[(2R,3S)-1-(tert-butoxycarbonyl)-2-methylazetidin-3-yl]oxy}-6-fluoropyridine-2-carboxylic acid (600 mg, 1.839 mmol, 1 equiv), HATU (1.05 g, 2.644 mmol, 1.5 equiv), DIEA (1.19 g, 8.815 mmol, 5 equiv), and DMF (6 mL) at room temperature. The resulting mixture was stirred for 0.5 h at room temperature under nitrogen atmosphere. To the above mixture was added aminocyclopropane (125.98 mg, 2.207 mmol, 1.2 equiv) in portions over 1 min at room temperature. The resulting mixture was stirred for additional 1 h at room temperature. Desired product could be detected by LCMS. The resulting mixture was extracted with EtOAc (3×20 mL). The combined organic layers were washed with brine (3×20 mL) and dried over anhydrous Na₂SO₄. After filtration, the filtrate was concentrated under reduced pressure. The resulting mixture was concentrated under reduced pressure. This resulted in tert-butyl (2R,3S)-3-{[6-(cyclopropylcarbamoyl)-2-fluoropyridin-3-yl]oxy}-2-methylazetidine-1-carboxylate (650 mg, 96.75%).

LC-MS: (ES+H, m/z): [M+H]⁺=366.

### Step 2: Preparation of N-cyclopropyl-6-fluoro-5-{[(2R,3S)-2-methylazetidin-3-yl]oxy}pyridine-2-carboxamide, TFA salt

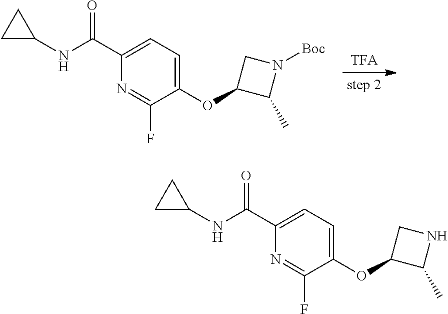 [SMILES: C[C@@H]1[C@@H](Oc2ccc(C(=O)NC3CC3)nc2F)CN1C(=O)OC(C)(C)C ; C[C@H]1NC[C@@H]1Oc1ccc(C(=O)NC2CC2)nc1F]

Into a 40 mL vial were added tert-butyl (2R,3S)-3-{[6-(cyclopropylcarbamoyl)-2-fluoropyridin-3-yl]oxy}-2-methylazetidine-1-carboxylate (650 mg, 1.916 mmol, 1 equiv) and DCM (6 mL) at room temperature. To the above mixture was added trifluoroacetic acid (4 mL, 0.005 mmol, 0.17 equiv) dropwise over 3 min at 0° C. The resulting mixture was stirred for 1 h at room temperature under air atmosphere. Desired product could be detected by LCMS. The resulting mixture was concentrated under reduced pressure. The crude product was used in the next step directly without further purification. This resulted in N-cyclopropyl-6-fluoro-5-{[(2R,3S)-2-methylazetidin-3-yl]oxy}pyridine-2-carboxamide, TFA salt (640 mg, crude).

LC-MS: (ES+H, m/z): [M+H]⁺=266.

### Step 3: Preparation of N-cyclopropyl-6-fluoro-5-{[(2R,3S)-1-({6-fluoro-4-oxo-2H,3H,5H-furo[3,2-c]quinolin-7-yl}methyl)-2-methylazetidin-3-yl]oxy}pyridine-2-carboxamide

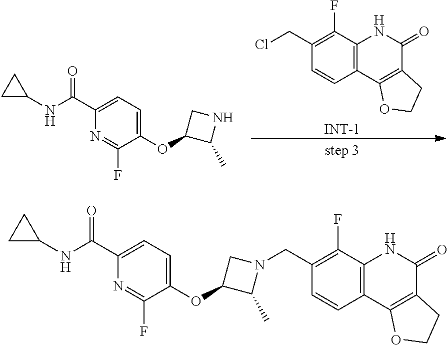 [SMILES: C[C@H]1NC[C@@H]1Oc1ccc(C(=O)NC2CC2)nc1F ; O=c1[nH]c2c(F)c(CCl)ccc2c2c1CCO2 ; "INT-1" ; C[C@@H]1[C@@H](Oc2ccc(C(=O)NC3CC3)nc2F)CN1Cc1ccc2c3c(c(=O)[nH]c2c1F)CCO3]

Into a 40 mL vial were added N-cyclopropyl-6-fluoro-5-{[(2R,3S)-2-methylazetidin-3-yl]oxy}pyridine-2-carboxamide (600 mg, 2.262 mmol, 1 equiv), 7-(chloromethyl)-6-fluoro-2H,3H,5H-furo[3,2-c]quinolin-4-one (688.44 mg, 2.714 mmol, 1.2 equiv), DIEA (1.46 g, 11.310 mmol, 5 equiv), KI (75.09 mg, 0.452 mmol, 0.2 equiv), and ACN (6 mL) at room temperature. The resulting mixture was stirred for 2 h at room temperature under nitrogen atmosphere. Desired product could be detected by LCMS. The precipitated solids were collected by filtration and washed with ACN (3×30 mL). The crude product (400 mg) was purified by Prep-HPLC with the following conditions (Column: YMC-Actus Triart C18 EXRS 30*150 mm, 5 um; Mobile Phase A: Water (10 mmol/L NH₄HCO₃+0.05% NH₃H₂O), Mobile Phase B: ACN; Flow rate: 100 mL/min; Gradient: 25% B to 55% B in 30 min; Wave Length: 254 nm/220 nm; RT1 (min): 9.88) to afford N-cyclopropyl-6-fluoro-5-{[(2R,3S)-1-({6-fluoro-4-oxo-2H,3H,5H-furo[3,2-c]quinolin-7-yl}methyl)-2-methylazetidin-3-yl]oxy}pyridine-2-carboxamide (102 mg, 9.35%).

LC-MS: (ES+H, m/z): [M+H]⁺=483.

¹H NMR (400 MHZ, DMSO-d₆) δ 11.39 (s, 1H), 8.43 (d, J=4.9 Hz, 1H), 7.85 (d, J=8.2 Hz, 1H), 7.60 (dd, J=10.1, 8.3 Hz, 1H), 7.38 (d, J=8.1 Hz, 1H), 7.20 (dd, J=8.2, 6.2 Hz, 1H), 4.82 (t, J=9.3 Hz, 2H), 4.59 (q, J=5.9 Hz, 1H), 4.07-3.58 (m, 3H), 3.47 (d, J=21.1 Hz, 1H), 3.42 (s, 2H), 3.39 (t, J=6.1 Hz, 2H), 2.84 (ddd, J=16.3, 8.2, 5.2 Hz, 3H), 0.71-0.59 (m, 4H).

¹⁹F NMR (377 MHz, DMSO-d₆) δ 84.57, −133.56.

### Example 4: Synthesis of N-(2,2-difluoroethyl)-6-fluoro-5-{[(2R,3S)-1-({6-fluoro-4-oxo-2H,3H,5H-furo[3,2-c]quinolin-7-yl}methyl)-2-methylazetidin-3-yl]oxy}pyridine-2-carboxamide (Compound 4)

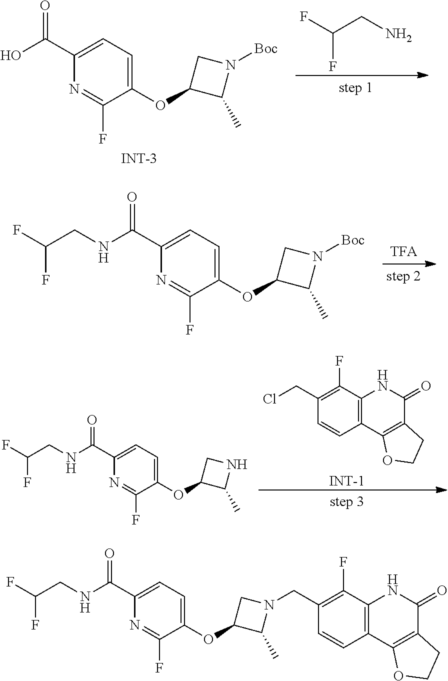

### Step 1: Preparation of tert-butyl (2R,3S)-3-({6-[(2,2-difluoroethyl) carbamoyl]-2-fluoropyridin-3-yl}oxy)-2-methylazetidine-1-carboxylate

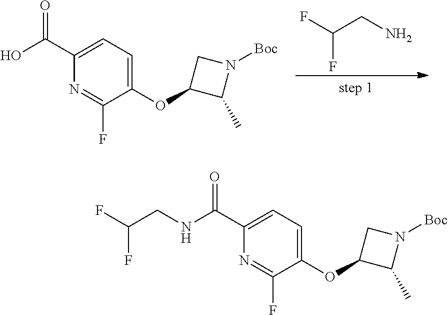 [SMILES: C[C@@H]1[C@@H](Oc2ccc(C(=O)O)nc2F)CN1C(=O)OC(C)(C)C ; NCC(F)F ; C[C@@H]1[C@@H](Oc2ccc(C(=O)NCC(F)F)nc2F)CN1C(=O)OC(C)(C)C]

Into a 40 mL vial were added 5-{[(2R,3S)-1-(tert-butoxycarbonyl)-2-methylazetidin-3-yl]oxy}-6-fluoropyridine-2-carboxylic acid (600 mg, 1.839 mmol, 1 equiv), HATU (1.05 g, 2.758 mmol, 1.5 equiv), DIEA (1.19 g, 9.195 mmol, 5 equiv), and DMF (6 mL) at room temperature. The resulting mixture was stirred for 0.5 h at room temperature under nitrogen atmosphere. To the above mixture was added 2,2-difluoroethanamine (163.96 mg, 2.023 mmol, 1.1 equiv) in portions over 3 min at room temperature. The resulting mixture was stirred for additional 1.5 h at room temperature. Desired product could be detected by LCMS. The resulting mixture was extracted with EtOAc (3×30 mL). The combined organic layers were washed with brine (3×30 mL) and dried over anhydrous Na₂SO₄. After filtration, the filtrate was concentrated under reduced pressure. This resulted in tert-butyl (2R,3S)-3-({6-[(2,2-difluoroethyl) carbamoyl]-2-fluoropyridin-3-yl}oxy)-2-methylazetidine-1-carboxylate (500 mg, 69.84%).

LC-MS: (ES+H, m/z): [M+H]⁺=390.

### Step 2: Preparation of N-(2,2-difluoroethyl)-6-fluoro-5-{[(2R,3S)-2-methylazetidin-3-yl]oxy}pyridine-2-carboxamide, TFA salt

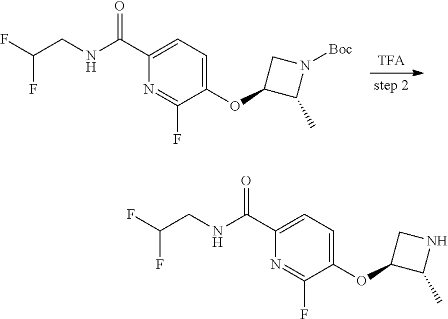 [SMILES: C[C@@H]1[C@@H](Oc2ccc(C(=O)NCC(F)F)nc2F)CN1C(=O)OC(C)(C)C ; C[C@H]1NC[C@@H]1Oc1ccc(C(=O)NCC(F)F)nc1F]

Into a 40 mL vial were added tert-butyl (2R,3S)-3-({6-[(2,2-difluoroethyl) carbamoyl]-2-fluoropyridin-3-yl}oxy)-2-methylazetidine-1-carboxylate (480 mg, 1.233 mmol, 1 equiv) and DCM (5 mL, 7.865 mmol) at room temperature. To the above mixture was added trifluoroacetic acid (5 mL, 0.005 mmol, 0.2 equiv) dropwise over 3 min at 0° C. The resulting mixture was stirred for 1 h at room temperature under air atmosphere. Desired product could be detected by LCMS. The resulting mixture was concentrated under reduced pressure. The crude product was used in the next step directly without further purification. This resulted in N-(2,2-difluoroethyl)-6-fluoro-5-{[(2R,3S)-2-methylazetidin-3-yl]oxy}pyridine-2-carboxamide, TFA salt (480 mg, crude).

LC-MS: (ES+H, m/z): [M+H]⁺=290.

### Step 3: Preparation of N-(2,2-difluoroethyl)-6-fluoro-5-{[(2R,3S)-1-({6-fluoro-4-oxo-2H,3H,5H-furo[3,2-c]quinolin-7-yl}methyl)-2-methylazetidin-3-yl]oxy}pyridine-2-carboxamide

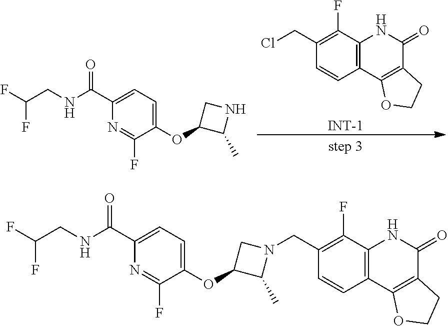 [SMILES: C[C@H]1NC[C@@H]1Oc1ccc(C(=O)NCC(F)F)nc1F ; O=c1[nH]c2c(F)c(CCl)ccc2c2c1CCO2 ; "INT-1" ; C[C@@H]1[C@@H](Oc2ccc(C(=O)NCC(F)F)nc2F)CN1Cc1ccc2c3c(c(=O)[nH]c2c1F)CCO3]

Into a 40 mL vial were added N-(2,2-difluoroethyl)-6-fluoro-5-{[(2R,3S)-2-methylazetidin-3-yl]oxy}pyridine-2-carboxamide (400 mg, 1.383 mmol, 1 equiv), 7-(chloromethyl)-6-fluoro-2H,3H,5H-furo[3,2-c]quinolin-4-one (420.93 mg, 1.660 mmol, 1.2 equiv), DIEA (893.65 mg, 6.915 mmol, 5 equiv), KI (45.91 mg, 0.277 mmol, 0.2 equiv) and ACN (4 mL) at room temperature. The resulting mixture was stirred for 2 h at 80° C. under nitrogen atmosphere. Desired product could be detected by LCMS. The precipitated solids were collected by filtration and washed with ACN (3×30 mL). The crude product (350 mg) was purified by Prep-HPLC with the following conditions (Column: YMC-Actus Triart C18 EXRS 30*150 mm, 5 um; Mobile Phase A: Water (10 mmol/L NH₄HCO₃+0.05% NH₃H₂O), Mobile Phase B: MeOH; Flow rate: 100 mL/min; Gradient: 30% B to 60% B in 30 min; Wave Length: 254 nm/220 nm; RT1 (min): 9.88) to afford N-(2,2-difluoroethyl)-6-fluoro-5-{[(2R,3S)-1-({6-fluoro-4-oxo-2H,3H,5H-furo[3,2-c]quinolin-7-yl}methyl)-2-methylazetidin-3-yl]oxy}pyridine-2-carboxamide (102 mg, 14.56%).

LC-MS: (ES+H, m/z): [M+H]⁺=507.

¹H NMR (400 MHZ, DMSO-d₆) δ 11.39 (s, 1H), 8.80 (t, J=6.2 Hz, 1H), 7.89 (d, J=8.2 Hz, 1H), 7.63 (dd, J=10.1, 8.2 Hz, 1H), 7.39 (d, J=8.1 Hz, 1H), 7.20 (dd, J=8.2, 6.2 Hz, 1H), 6.11 (t, J=4.1 Hz, 1H), 4.82 (t, J=9.3 Hz, 2H), 4.62 (q, J=5.9 Hz, 1H), 3.95-3.87 (m, 1H), 3.82 (t, J=6.4 Hz, 1H), 3.66 (dddd, J=19.3, 15.2, 10.1, 3.8 Hz, 3H), 3.41 (q, J=6.0 Hz, 1H), 3.07 (t, J=9.3 Hz, 2H), 2.83 (t, J=6.7 Hz, 1H), 1.19 (d, J=6.1 Hz, 3H).

¹⁹F NMR (377 MHZ, DMSO-d₆) δ 84.47, −121.93, −133.54.

### Example 5: Synthesis of 6-fluoro-5-(((2R,3S)-1-((5-fluoro-2-methyl-3-oxo-3,4-dihydroquinoxalin-6-yl)methyl)-2-methylazetidin-3-yl)oxy)-N-methylpicolinamide (Compound 5)

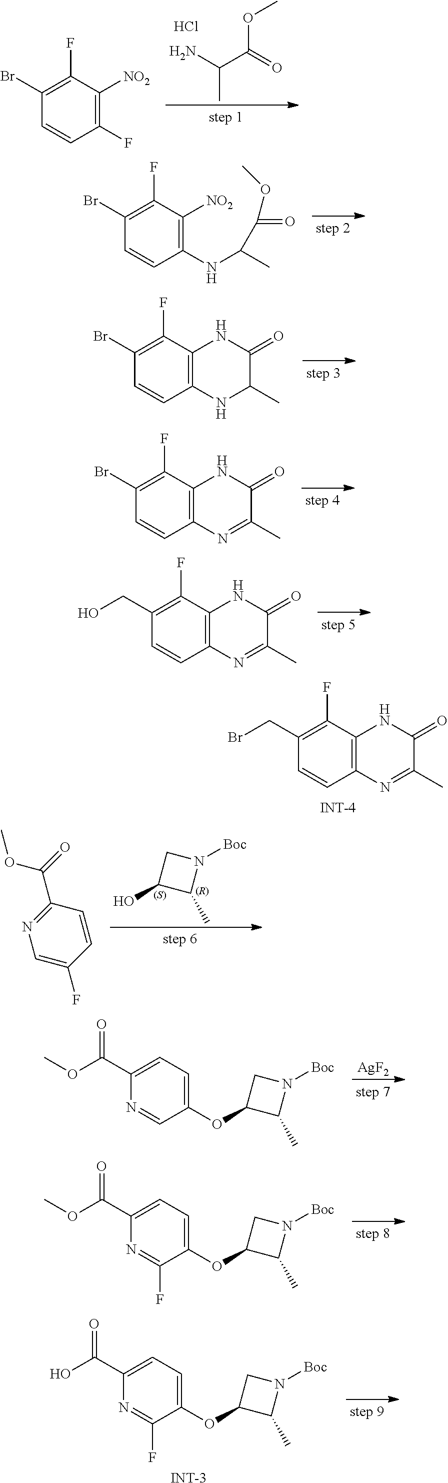

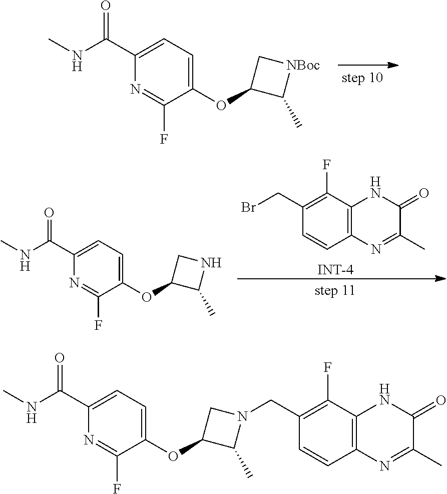

### Step 1: Preparation of methyl 2-[(4-bromo-3-fluoro-2-nitrophenyl)amino]propanoate

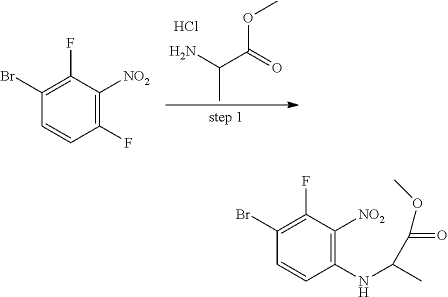

Into a 250 mL round-bottom flask were added methyl 2-aminopropanoate hydrochloride (14.66 g, 105.047 mmol, 1.00 equiv), DIEA (40.73 g, 315.141 mmol, 3 equiv), 1-bromo-2,4-difluoro-3-nitrobenzene (25 g, 105.047 mmol, 1.00 equiv), and DMF (125 mL) at room temperature. The resulting mixture was stirred for overnight at room temperature under nitrogen atmosphere. Desired product could be detected by LCMS. The resulting mixture was diluted with water (2 L). The resulting mixture was extracted with EtOAc (3×2 L). The combined organic layers were washed with brine (1×2 L) and dried over anhydrous Na₂SO₄. After filtration, the filtrate was concentrated under reduced pressure. The residue was purified by trituration with CH₂Cl₂:Hexane (1:10) (160 mL). The resulting solid was dried under vacuum. This resulted in methyl 2-[(4-bromo-3-fluoro-2-nitrophenyl)amino]propanoate (20 g, 59.29%).

LC-MS: (ES+H, m/z): [M+H]⁺=321.

### Step 2: Preparation of 7-bromo-8-fluoro-3-methyl-3,4-dihydro-1H-quinoxalin-2-one

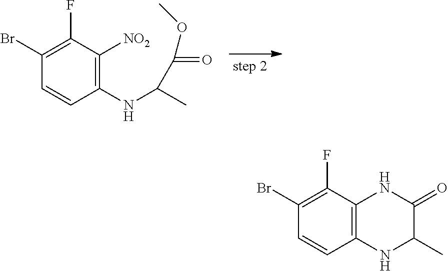 [SMILES: COC(=O)C(C)Nc1ccc(Br)c(F)c1[N+](=O)[O-] ; CC1Nc2ccc(Br)c(F)c2NC1=O]

Into a 500 mL round-bottom flask were added methyl 2-[(4-bromo-3-fluoro-2-nitrophenyl)amino]propanoate (20 g, 80.971 mmol, 1 equiv), Fe (17.44 g, 404.855 mmol, 5 equiv), and CH₃COOH (200 mL) at room temperature. The resulting mixture was stirred for 3 h at 80° C. under nitrogen atmosphere. Desired product could be detected by LCMS. The mixture was allowed to cool down to room temperature. The resulting mixture was diluted with water (150 mL). The mixture was basified to pH 8 with saturated NaHCO₃ (aq.). The resulting mixture was extracted with CH₂Cl₂:2-propanol=5:1 (3×1.2 L). The combined organic layers were washed with brine (1×3 L) and dried over anhydrous Na₂SO₄. After filtration, the filtrate was concentrated under reduced pressure. This resulted in 7-bromo-8-fluoro-3-methyl-3,4-dihydro-1H-quinoxalin-2-one (10.4 g, 49.42%).

LC-MS: (ES+H, m/z): [M+H]⁺=259.

### Step 3: Preparation of 7-bromo-8-fluoro-3-methyl-1H-quinoxalin-2-one

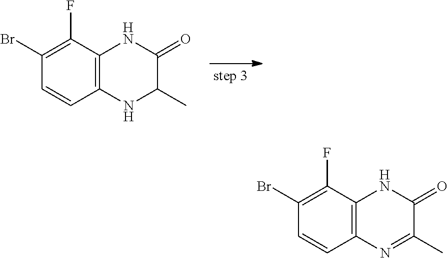

Into a 1 L round-bottom flask were added 7-bromo-8-fluoro-3-methyl-3,4-dihydro-1H-quinoxalin-2-one (10 g, 38.598 mmol, 1 equiv), DDQ (10514.21 mg, 46.318 mmol, 1.20 equiv), and CH₂Cl₂ (300 mL) at room temperature. The resulting mixture was stirred for 2.5 h at room temperature under nitrogen atmosphere. Desired product could be detected by LCMS. The resulting mixture was concentrated under reduced pressure. The resulting mixture was diluted with water (100 mL). The mixture was neutralized to pH 7 with saturated NaHCO₃ (aq.). The precipitated solids were collected by filtration and washed with water (3×1 L). The crude product was used in the next step directly without further purification. This resulted in 7-bromo-8-fluoro-3-methyl-1H-quinoxalin-2-one (5 g, 50.21%).

LC-MS: (ES+H, m/z): [M+H]⁺=257.

### Step 4: Preparation of 8-fluoro-7-(hydroxymethyl)-3-methyl-1H-quinoxalin-2-one

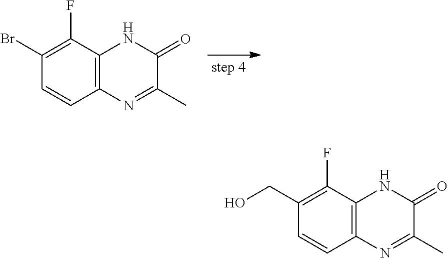

Into a 500 mL 3-necked round-bottom flask were added 7-bromo-8-fluoro-3-methyl-1H-quinoxalin-2-one (4.9 g, 27.231 mmol, 1 equiv), (tributylstannyl) methanol (7.34 g, 32.677 mmol, 1.2 equiv), 2nd Generation XPhos Precatalyst (1.53 g, 2.723 mmol, 0.10 equiv), and 1,4-dioxane (100 mL) at room temperature. The resulting mixture was stirred for 2 h at 80° C. under nitrogen atmosphere. Desired product could be detected by LCMS. The resulting mixture was concentrated under reduced pressure. The residue was purified by silica gel column chromatography, eluted with CH₂Cl₂/MeOH (9:1) to afford 8-fluoro-7-(hydroxymethyl)-3-methyl-1H-quinoxalin-2-one (2.6 g, 76.44%).

LC-MS: (ES+H, m/z): [M+H]⁺=209.

### Step 5: Preparation of 7-(bromomethyl)-8-fluoro-3-methyl-1H-quinoxalin-2-one

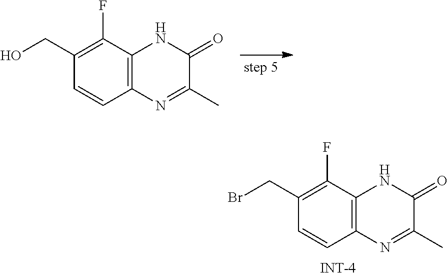

To a stirred solution of 8-fluoro-7-(hydroxymethyl)-3-methyl-1H-quinoxalin-2-one (2.5 g, 24.016 mmol, 1 equiv) in CH₂Cl₂ (50 mL) was added PBr₃ (7.50 g, 48.032 mmol, 2 equiv) dropwise at 0° C. under nitrogen atmosphere. The resulting mixture was stirred for 3 h at 50° C. under nitrogen atmosphere. Desired product could be detected by LCMS. The resulting mixture was concentrated under reduced pressure. The residue was purified by trituration with diethyl ether (50 mL). This resulted in 7-(bromomethyl)-8-fluoro-3-methyl-1H-quinoxalin-2-one (2.5 g, crude).

LC-MS: (ES+H, m/z): [M+H]⁺=271.

### Step 6: Preparation of methyl 5-{[(2R,3S)-1-(tert-butoxycarbonyl)-2-methylazetidin-3-yl]oxy}pyridine-2-carboxylate

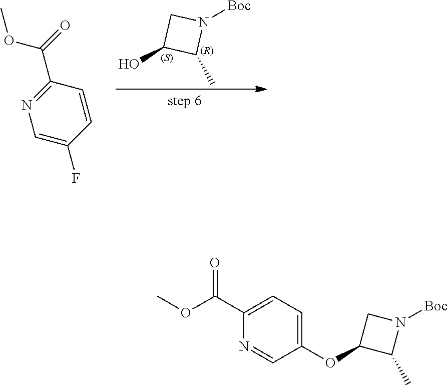

Into a 250 mL round-bottom flask were added methyl 5-fluoropyridine-2-carboxylate (10 g, 64.463 mmol, 1 equiv), K₂CO₃ (22.27 g, 161.157 mmol, 2.5 equiv), tert-butyl (2R,3S)-3-hydroxy-2-methylazetidine-1-carboxylate (13.88 g, 74.132 mmol, 1.15 equiv), and DMSO (100 mL) at room temperature. The resulting mixture was stirred for 3 h at 100° C. under nitrogen atmosphere. Desired product could be detected by LCMS. The resulting mixture was diluted with water (100 mL). The resulting mixture was extracted with EtOAc (3×200 mL). The combined organic layers were washed with brine (1×200 mL) and dried over anhydrous Na₂SO₄. After filtration, the filtrate was concentrated under reduced pressure. The residue was purified by silica gel column chromatography, eluted with PE/EA (3:1), to afford methyl 5-{[(2R,3S)-1-(tert-butoxycarbonyl)-2-methylazetidin-3-yl]oxy}pyridine-2-carboxylate (12 g, 57.75%).

LC-MS: (ES+H, m/z): [M+H]⁺=323.

### Step 7: Preparation of methyl 5-{[(2R,3S)-1-(tert-butoxycarbonyl)-2-methylazetidin-3-yl]oxy}-6-fluoropyridine-2-carboxylate

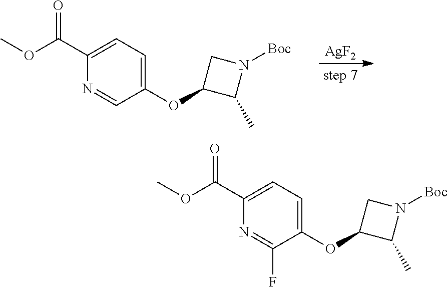

Into a 1000 mL vial were added methyl 5-{[(2R,3S)-1-(tert-butoxycarbonyl)-2-methylazetidin-3-yl]oxy}pyridine-2-carboxylate (10 g, 31.021 mmol, 1 equiv), difluorosilver (22.62 g, 155.105 mmol, 5 equiv), and ACN (325 mL) at room temperature. The resulting mixture was stirred for 2 days at 40° C. under nitrogen atmosphere. Desired product could be detected by LCMS. The resulting mixture was filtered, and the filter cake was washed with ACN (3×50 mL). The filtrate was concentrated under reduced pressure. The residue was purified by silica gel column chromatography, eluted with PE/EA (2:1), to afford methyl 5-{[(2R,3S)-1-(tert-butoxycarbonyl)-2-methylazetidin-3-yl]oxy}-6-fluoropyridine-2-carboxylate (2.5 g, 17.84%).

LC-MS: (ES+H, m/z): [M+H]⁺=341.

### Step 8: Preparation of 5-{[(2R,3S)-1-(tert-butoxycarbonyl)-2-methylazetidin-3-yl]oxy}-6-fluoropyridine-2-carboxylic acid

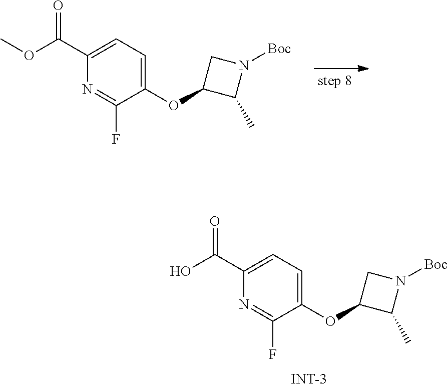

Into a 250 mL round-bottom flask were added methyl 5-{[(2R,3S)-1-(tert-butoxycarbonyl)-2-methylazetidin-3-yl]oxy}-6-fluoropyridine-2-carboxylate (2.4 g, 23.505 mmol, 1 equiv), LiOH (843 mg, 117.525 mmol, 5 equiv), and THF (20 mL), H₂O (5 mL) at room temperature. The resulting mixture was stirred for 1 h at room temperature under nitrogen atmosphere. Desired product could be detected by LCMS. The mixture was acidified to pH 4 with conc. HCl. The resulting mixture was extracted with EtOAc (3×50 mL). The combined organic layers were washed with brine (1×100 mL) and dried over anhydrous Na₂SO₄. After filtration, the filtrate was concentrated under reduced pressure. This resulted in 5-{[(2R,3S)-1-(tert-butoxycarbonyl)-2-methylazetidin-3-yl]oxy}-6-fluoropyridine-2-carboxylic acid (1.8 g, 93.87%).

LC-MS: (ES+H, m/z): [M+H]⁺=327.

### Step 9: Preparation of tert-butyl (2R,3S)-3-{[2-fluoro-6-(methylcarbamoyl)pyridin-3-yl]oxy}-2-methylazetidine-1-carboxylate

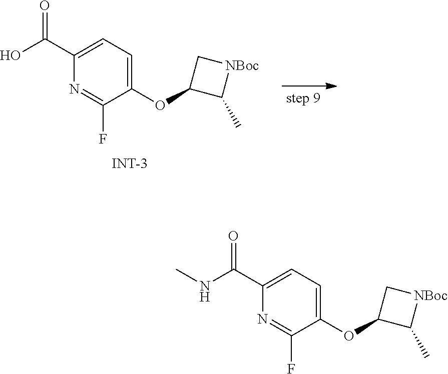

Into a 40 mL vial were added 5-{[(2R,3S)-1-(tert-butoxycarbonyl)-2-methylazetidin-3-yl]oxy}-6-fluoropyridine-2-carboxylic acid (800 mg, 2.758 mmol, 1 equiv), HATU (1.39 g, 4.137 mmol, 1.5 equiv), DIEA (1.58 g, 13.790 mmol, 5 equiv), and DMF (8 mL, 12.904 mmol) at room temperature. The resulting mixture was stirred for 0.5 h at room temperature under nitrogen atmosphere. To the above mixture was added CH₃NH₂HCl (198.63 mg, 3.310 mmol, 1.2 equiv) in portions over 1 min at room temperature. The resulting mixture was stirred for additional 1 h at room temperature. Desired product could be detected by LCMS. The resulting mixture was extracted with EtOAc (3×50 mL). The combined organic layers were washed with brine (1×100 mL) and dried over anhydrous Na₂SO₄. After filtration, the filtrate was concentrated under reduced pressure. The residue was purified by silica gel column chromatography, eluted with PE/EA (1:1), to afford tert-butyl (2R,3S)-3-{[2-fluoro-6-(methylcarbamoyl)pyridin-3-yl]oxy}-2-methylazetidine-1-carboxylate (650 mg, 86.16%).

LC-MS: (ES+H, m/z): [M+H]⁺=340.

### Step 10: Preparation of 6-fluoro-N-methyl-5-{[(2R,3S)-2-methylazetidin-3-yl]oxy}pyridine-2-carboxamide, TFA salt

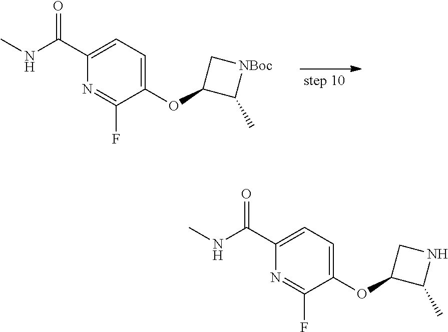

Into a 40 mL vial were added tert-butyl (2R,3S)-3-{[2-fluoro-6-(methylcarbamoyl)pyridin-3-yl]oxy}-2-methylazetidine-1-carboxylate (600 mg, 0.029 mmol, 1 equiv), DCM (6 mL), and trifluoroacetaldehyde (6 mL, 0.005 mmol, 0.17 equiv) at room temperature. The resulting mixture was stirred for 2 h at room temperature under air atmosphere. Desired product could be detected by LCMS. The resulting mixture was concentrated under reduced pressure. The crude product was used in the next step directly without further purification. This resulted in 6-fluoro-N-methyl-5-{[(2R,3S)-2-methylazetidin-3-yl]oxy}pyridine-2-carboxamide, TFA salt (700 mg, crude).

LC-MS: (ES+H, m/z): [M+H]⁺=240.

### Step 11: Preparation of 6-fluoro-5-{[(2R,3S)-1-[(5-fluoro-2-methyl-3-oxo-4H-quinoxalin-6-yl)methyl]-2-methylazetidin-3-yl]oxy}-N-methylpyridine-2-carboxamide

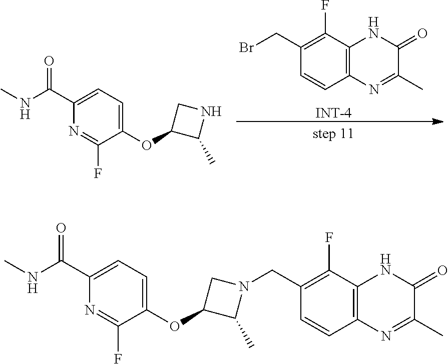

Into a 20 mL vial were added 6-fluoro-N-methyl-5-{[(2R,3S)-2-methylazetidin-3-yl]oxy}pyridine-2-carboxamide, TFA salt (500 mg, 2.090 mmol, 1 equiv), 7-(bromomethyl)-8-fluoro-3-methyl-1H-quinoxalin-2-one (679.85 mg, 2.508 mmol, 1.2 equiv), DIEA (1.35 g, 10.450 mmol, 5 equiv), KI (69.38 mg, 0.418 mmol, 0.2 equiv), and CH₃CN (5 mL) at room temperature. The resulting mixture was stirred for 2 h at 80° C. under nitrogen atmosphere. Desired product could be detected by LCMS. The residue was purified by trituration with CH₃CN (10 mL). The crude product (300 mg) was purified by Prep-HPLC with the following conditions (Column: MIX/ACN-Ph C18 EXRS 50*250 mm, 10 μm; Mobile Phase A: Water (10 mmol/L NH₄HCO₃+0.05% NH₃H₂O), Mobile Phase B: ACN; Flow rate: 100 mL/min mL/min; Gradient: 10% B to 40% B in 30 min; Wave Length: 254 nm/220 nm; RT1 (min): 9.8) to afford 6-fluoro-5-{[(2R,3S)-1-[(5-fluoro-2-methyl-3-oxo-4H-quinoxalin-6-yl)methyl]-2-methylazetidin-3-yl]oxy}-N-methylpyridine-2-carboxamide (102 mg, 11.30%).

LC-MS: (ES+H, m/z): [M+H]⁺=430.

¹H NMR (300 MHz, DMSO-d₆) δ 12.46 (s, 1H), 8.49 (d, J=5.0 Hz, 1H), 7.85 (d, J=8.1 Hz, 1H), 7.61 (dd, J=10.2, 8.2 Hz, 1H), 7.49 (d, J=8.3 Hz, 1H), 7.26 (t, J=7.6 Hz, 1H), 4.60 (q, J=5.9 Hz, 1H), 3.91 (d, J=13.2 Hz, 2H), 3.82 (t, J=6.4 Hz, 1H), 3.69 (d, J=13.1 Hz, 1H), 2.76 (d, J=4.7 Hz, 4H), 2.41 (s, 3H), 1.20 (d, J=6.2 Hz, 3H).

¹⁹F NMR (282 MHZ, DMSO-d₆) δ −84.71, −136.07.

### Example 6: Synthesis of N-cyclopropyl-6-fluoro-5-(((2R,3S)-1-((5-fluoro-2-methyl-3-oxo-3,4-dihydroquinoxalin-6-yl)methyl)-2-methylazetidin-3-yl)oxy) picolinamide (Compound 6)

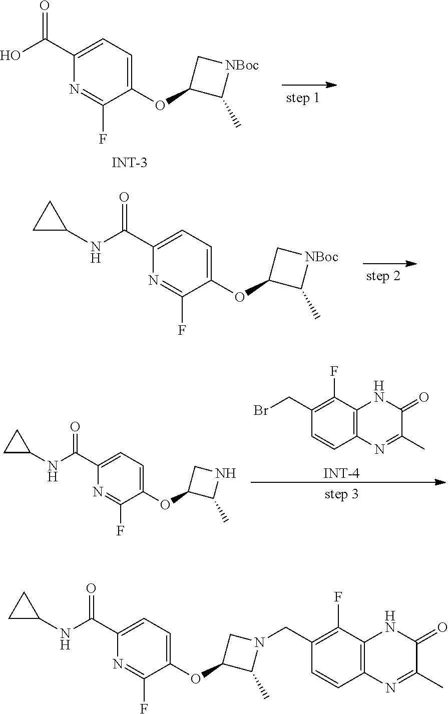

### Step 1: Preparation of tert-butyl (2R,3S)-3-{[6-(cyclopropylcarbamoyl)-2-fluoropyridin-3-yl]oxy}-2-methylazetidine-1-carboxylate

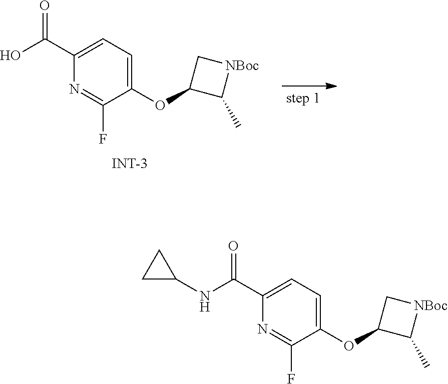

Into a 40 mL vial were added 5-{[(2R,3S)-1-(tert-butoxycarbonyl)-2-methylazetidin-3-yl]oxy}-6-fluoropyridine-2-carboxylic acid (800 mg, 2.758 mmol, 1 equiv), HATU (1.39 g, 4.137 mmol, 1.5 equiv), DIEA (1.58 g, 13.790 mmol, 5 equiv), and DMF (8 mL) at room temperature. To the above mixture was added aminocyclopropane (167.96 mg, 3.310 mmol, 1.2 equiv) in portions over 1 min at room temperature. The resulting mixture was stirred for additional 1 h at room temperature. Desired product could be detected by LCMS. The resulting mixture was extracted with EtOAc (3×50 mL). The combined organic layers were washed with brine (1×100 mL) and dried over anhydrous Na₂SO₄. After filtration, the filtrate was concentrated under reduced pressure. The residue was purified by silica gel column chromatography, eluted with PE/EA (1:1), to afford tert-butyl (2R,3S)-3-{[6-(cyclopropylcarbamoyl)-2-fluoropyridin-3-yl]oxy}-2-methylazetidine-1-carboxylate (700 mg, 84.27%).

LC-MS: (ES+H, m/z): [M+H]⁺=366.

### Step 2: Preparation of N-cyclopropyl-6-fluoro-5-{[(2R,3S)-2-methylazetidin-3-yl]oxy}pyridine-2-carboxamide, TFA salt

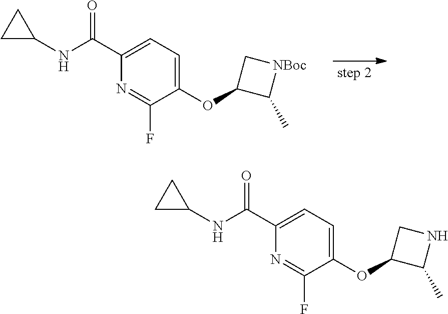 [SMILES: C[C@@H]1[C@@H](Oc2ccc(C(=O)NC3CC3)nc2F)CN1C(=O)OC(C)(C)C ; C[C@H]1NC[C@@H]1Oc1ccc(C(=O)NC2CC2)nc1F]

Into a 40 mL vial were added tert-butyl (2R,3S)-3-{[6-(cyclopropylcarbamoyl)-2-fluoropyridin-3-yl]oxy}-2-methylazetidine-1-carboxylate (650 mg, 0.027 mmol, 1 equiv), trifluoroacetaldehyde (6 mL), and DCM (6 mL) at room temperature. The resulting mixture was stirred for 2 h at room temperature under air atmosphere. Desired product could be detected by LCMS. The resulting mixture was concentrated under reduced pressure. The crude product was used in the next step directly without further purification. This resulted in N-cyclopropyl-6-fluoro-5-{[(2R,3S)-2-methylazetidin-3-yl]oxy}pyridine-2-carboxamide, TFA salt (680 g, crude).

LC-MS: (ES+H, m/z): [M+H]⁺=266.

### Step 3: Preparation of 7-bromo-8-fluoro-3-methyl-1H-quinoxalin-2-one

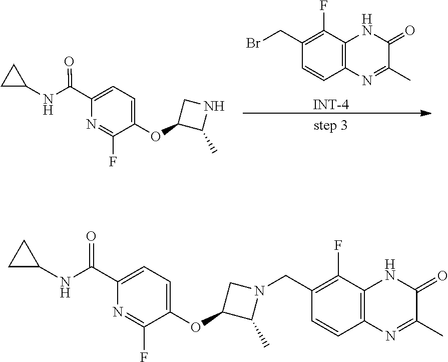

Into a 20 mL vial were added N-cyclopropyl-6-fluoro-5-{[(2R,3S)-2-methylazetidin-3-yl]oxy}pyridine-2-carboxamide, TFA salt (500 mg, 1.885 mmol, 1 equiv), DIEA (1.22 g, 9.425 mmol, 5 equiv), 7-(bromomethyl)-8-fluoro-3-methyl-1H-quinoxalin-2-one (613.12 mg, 2.262 mmol, 1.2 equiv), KI (62.57 mg, 0.377 mmol, 0.20 equiv), and CH₃CN (5 mL, 237.830 mmol, 126.17 equiv) at room temperature. The resulting mixture was stirred for 3 h at 80° C. under nitrogen atmosphere. Desired product could be detected by LCMS. The residue was purified by trituration with CH₃CN (30 mL). The crude product (250 mg) was purified by Prep-HPLC with the following conditions (Column: ACN-XB C18 EXRS 30*150 mm, 5 μm; Mobile Phase A: Water (10 mmol/L NH₄HCO₃+0.05% NH₃H₂O), Mobile Phase B: ACN; Flow rate: 100 mL/min mL/min; Gradient: 15% B to 45% B in 30 min; Wave Length: 254 nm/220 nm; RT1 (min): 9.8) to afford N-cyclopropyl-6-fluoro-5-{[(2R,3S)-1-[(5-fluoro-2-methyl-3-oxo-4H-quinoxalin-6-yl)methyl]-2-methylazetidin-3-yl]oxy}pyridine-2-carboxamide (100 mg, 11.63%).

LC-MS: (ES+H, m/z): [M+H]⁺=456.

¹H NMR (300 MHz, DMSO-d₆) δ 12.46 (s, 1H), δ 8.45 (d, J=5.0 Hz, 1H), 7.85 (d, J=8.1 Hz, 1H), 7.60 (t, J=9.2 Hz, 1H), 7.49 (d, J=8.3 Hz, 1H), 7.25 (t, J=7.6 Hz, 1H), 4.59 (q, J=6.1 Hz, 1H), 3.91 (d, J=13.2 Hz, 1H), 3.81 (t, J=6.4 Hz, 1H), 3.69 (d, J=13.3 Hz, 1H), 2.84 (dt, J=13.0, 6.3 Hz, 2H), 2.41 (s, 3H), 1.21 (t, J=6.7 Hz, 3H), 0.66 (d, J=6.9 Hz, 4H).

¹⁹F NMR (282 MHZ, DMSO-d₆) δ −84.59, −136.07.

### Example 7:6-fluoro-5-(((2R,3S)-1-((5-fluoro-2-methyl-3-oxo-3,4-dihydroquinoxalin-6-yl)methyl)-2-methylazetidin-3-yl)oxy) picolinonitrile (Compound 7)

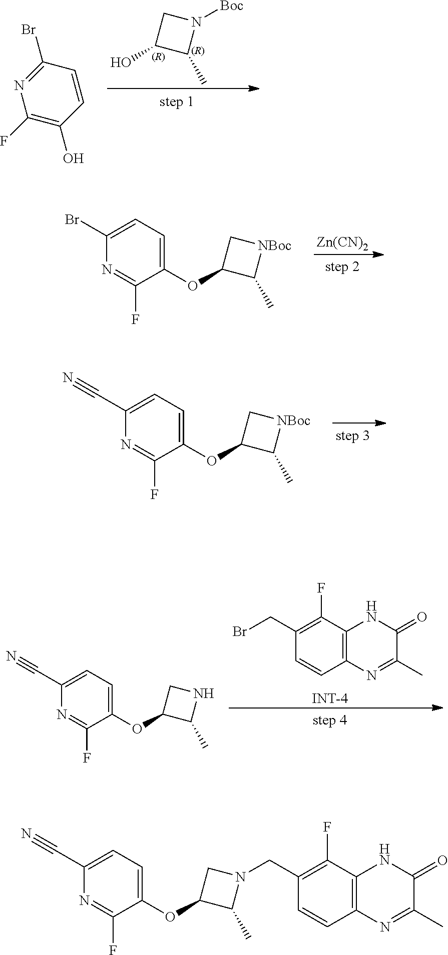

### Step 1: Preparation of tert-butyl (2R,3S)-3-[(6-bromo-2-fluoropyridin-3-yl)oxy]-2-methylazetidine-1-carboxylate

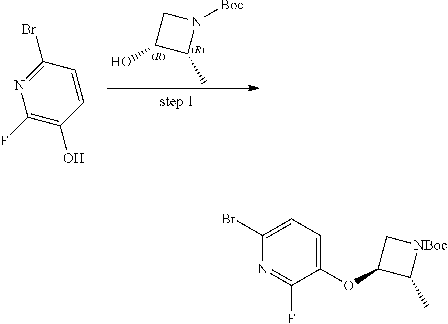

To a stirred solution of 6-bromo-2-fluoropyridin-3-ol (2 g, 10.417 mmol, 1 equiv) and tert-butyl (2S,3S)-3-hydroxy-2-methylazetidine-1-carboxylate (1.95 g, 10.417 mmol, 1 equiv) in THF (5 mL) were added DIAD (3.16 g, 15.625 mmol, 1.5 equiv) and PPh₃ (4.10 g, 15.625 mmol, 1.5 equiv) at room temperature under nitrogen atmosphere. The resulting mixture was stirred for overnight at 50° C. under nitrogen atmosphere. The reaction was monitored by LCMS. Desired product could be detected by LCMS. The resulting mixture was concentrated under reduced pressure. The residue was purified by silica gel column chromatography, eluted with PE/EA (1:1), to afford tert-butyl (2R,3S)-3-[(6-bromo-2-fluoropyridin-3-yl)oxy]-2-methylazetidine-1-carboxylate (2.2 g, 58.47%).

### Step 2: Preparation of tert-butyl 3-[(6-cyano-2-fluoropyridin-3-yl)oxy]-2-methylazetidine-1-carboxylate

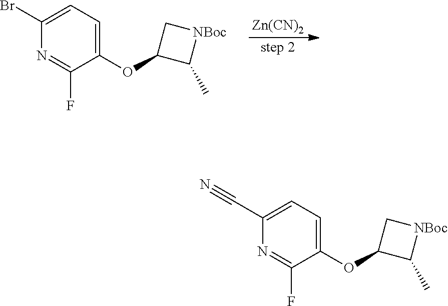

To a stirred solution of tert-butyl (2R,3S)-3-[(6-bromo-2-fluoropyridin-3-yl)oxy]-2-methylazetidine-1-carboxylate (2 g, 5.537 mmol, 1 equiv) and zinc dicarbonitrile (780.34 mg, 6.644 mmol, 1.2 equiv) in DMA (20 mL) were added dppf (611.69 mg, 1.107 mmol, 0.2 equiv) and Pd₂(dba)₃-CHCl₃ (573.13 mg, 0.554 mmol, 0.1 equiv) at room temperature under nitrogen atmosphere. The resulting mixture was stirred for 2 h at 100° C. under nitrogen atmosphere. The reaction was monitored by LCMS. Desired product could be detected by LCMS. The resulting mixture was concentrated under reduced pressure. The residue was purified by silica gel column chromatography, eluted with CH₂Cl₂/MeOH (10:1), to afford tert-butyl 3-[(6-cyano-2-fluoropyridin-3-yl)oxy]-2-methylazetidine-1-carboxylate (1.2 g, 70.52%).

### Step 3: Preparation of 6-fluoro-5-{[(2R,3S)-2-methylazetidin-3-yl]oxy}pyridine-2-carbonitrile

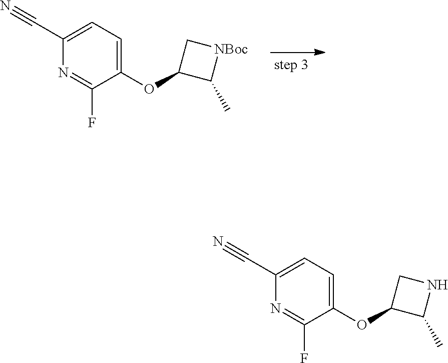 [SMILES: C[C@@H]1[C@@H](Oc2ccc(C#N)nc2F)CN1C(=O)OC(C)(C)C ; C[C@H]1NC[C@@H]1Oc1ccc(C#N)nc1F]

To a stirred solution of tert-butyl (2R,3S)-3-[(6-cyano-2-fluoropyridin-3-yl)oxy]-2-methylazetidine-1-carboxylate (100 mg, 0.325 mmol, 1 equiv) in DCM (5 mL) was added HCl (gas) in 1,4-dioxane (5 mL) at room temperature under nitrogen atmosphere. The reaction was monitored by LCMS. Desired product could be detected by LCMS. The resulting mixture was concentrated under reduced pressure. The crude product was used in the next step directly without further purification.

### Step 4: Preparation of 6-fluoro-5-{[(2R,3S)-1-[(5-fluoro-2-methyl-3-oxo-4H-quinoxalin-6-yl)methyl]-2-methylazetidin-3-yl]oxy}pyridine-2-carbonitrile

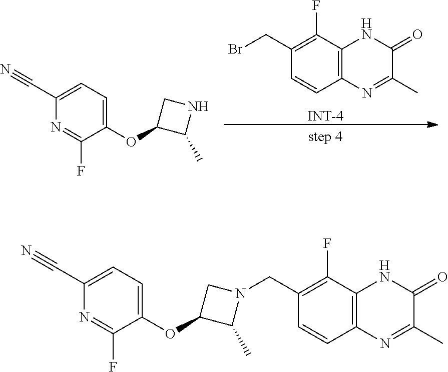

To a stirred solution of 6-fluoro-5-{[(2R,3S)-2-methylazetidin-3-yl]oxy}pyridine-2-carbonitrile hydrochloride (600 mg, 2.462 mmol, 1 equiv) and 7-(bromomethyl)-8-fluoro-3-methyl-1H-quinoxalin-2-one (801.02 mg, 2.954 mmol, 1.2 equiv) in ACN (5 mL) were added KI (81.75 mg, 0.492 mmol, 0.2 equiv) and DIEA (1591.25 mg, 12.310 mmol, 5 equiv) at room temperature under nitrogen atmosphere. The resulting mixture was stirred for 2 h at 80° C. under nitrogen atmosphere. The reaction was monitored by LCMS. The resulting mixture was concentrated under reduced pressure. The residue was purified by silica gel column chromatography, eluted with CH₂Cl₂/MeOH (10:1), to afford 6-fluoro-5-{[(2R,3S)-1-[(5-fluoro-2-methyl-3-oxo-4H-quinoxalin-6-yl)methyl]-2-methylazetidin-3-yl]oxy}pyridine-2-carbonitrile (100.5 mg, 10.25%).

LC-MS: (M+H⁺): 398.15.

¹H NMR (400 MHZ, DMSO-d₆) δ 12.43 (s, 1H), 7.97 (d, J=8.2 Hz, 1H), 7.65 (dd, J=10.0, 8.2 Hz, 1H), 7.49 (d, J=8.2 Hz, 1H), 7.25 (dd, J=8.3, 7.1 Hz, 1H), 4.66 (q, J=5.9 Hz, 1H), 3.95-3.87 (m, 1H), 3.82 (t, J=6.4 Hz, 1H), 3.73-3.65 (m, 1H), 3.41 (p, J=6.1 Hz, 1H), 2.82 (t, J=6.7 Hz, 1H), 2.41 (s, 3H), 1.19 (d, J=6.2 Hz, 3H).

Other compounds described herein can be made via similar procedures as those described above.

### Example A: Cell Growth Inhibition Assay

The objective of this study was to evaluate the effect of a compound disclosed herein on cell proliferation through the cell viability assay in DLD-1 BRCA2 (−/−) and parental isogenic pair and MDA-MB-436 (mutated BRCA1) cell lines. The CellTiter-Glo (CTG) based cell viability assay was designed to determine the number of viable cells in the culture because of compound effect, by quantifying adenosine triphosphate (ATP), which indicates the presence of metabolically active cells.

DLD-1 BRCA2 (−/−) and parental isogenic pair were cultured in RPMI 1640 supplemented with 10% fetal bovine serum (FBS), and MDA-MB-436 cells were cultured in Dulbecco's Modified Eagle Medium (DMEM) supplemented with 10% FBS. Both were cultured at 37° C. with 5% CO₂. Compounds were distributed to the 384 well plate (Corning, 3764) using Echo acoustic liquid handler to form a 1:3 serially diluted final concentration with top dose of 10 or 30 μM. The cells were seeded into the plate in the density of 50 cells/well (DLD-1 parental), 200 cells/well (DLD-1 BRCA2−/−), or 500 cells/well (MDA-MB-436). After a short spin, the cells were cultured in a well moisturized incubator at 37° C. with 5% CO₂ for 7 days without disturbance. The cell viability was measured by CellTiter Glo 2.0 assay kit (Promega, G9243), and growth inhibition rate was calculated and plotted against final compound concentration, and the data were fitted in Xfit to generate IC50.

### Example B: Biochemical (FP) Assay

Assays based on fluorescent polarization (FP) have been widely utilized in drug discovery due to the homogenous format, robust performance and lack of interference seen in other assays. To characterize compounds, an assay measuring the displacement of a commercially available fluorescently labeled PARP 1/2 inhibitor (PARPi-FL, Tocris Biosciences, #6461) was utilized as exemplified in assays performed in WO2014/064149 and WO2021/013735A1. The assay was performed utilizing the following method:

Compounds were dissolved in DMSO an Echo550 liquid handler was utilized to make serial dilations in the desired concentration range in Optiplate-384F plates. 100% DMSO was used for the high (with protein) and low (without protein) control samples. 20 nL of compound or DMSO alone was added to individual assay plate wells.

PARP1 and PARP2 protein were expressed, purified, and diluted in assay buffer containing 50 mM Tris pH 8.0, 0.001% Triton X-100, 10 mM MgCl₂, 150 mM NaCl to a final concentration of 20 nM. The PARPi-FL was then added at a final concentration of 3 nM.

The assay plate was centrifuged at 1000 rpm for 1 min and incubated for 4 h at room temperature.

The fluorescent polarization was read using an Envision plate reader using the following settings:
Excitation filter—FITC FP 480-Ex Slot 3
Emission filter—FITC FP P-pol 535-Em Slot 4
2nd Emission filter—FITC FP S-pol 535-Em Slot 3
Mirror module—FITC FP Dual Enh-Slot 1

The inhibition rate was calculated using the percentage of permuted Mahalanobis distances greater than the control samples (mP value) following the equation below:

mP_{c}: the mP value of compounds
mP_{L}: the mP value of Low controls
mP_{H}: the mP value of High controls

$$Inhibition ( \% ) = ( 1 - \frac{m {P}_{C} - m {P}_{L}}{m {P}_{H} - m {P}_{L}} ) \times 100 \%$$

XLFit (equation 201) was used to calculate a reported IC50 for each compound.

The data from Example A and Example B are provided in Table 2.

TABLE 2

| | | IC₅₀ | | | |
|---|---|---|---|---|---|
| | IC₅₀ DLD-1 | DLD-1 | IC₅₀ MDA- | IC₅₀ FP | IC₅₀ FP |
| Compound | BRCA2 | parental | MB-436 | PARP1 | PARP2 |
| No. | (−/−) μM | μM | μM | μM | μM |
| 1 | | | | 0.006 | >100 |
| 2 | | | | 0.008 | >100 |
| 3 | | | | 0.005 | >100 |
| 4 | | | | 0.010 | >100 |
| 5 | 0.067 | >10 | 0.023 | 0.014 | >100 |
| 6 | 0.028 | >10 | 0.0089 | 0.013 | >100 |
| 7 | >1 | >10 | >1 | 0.044 | >100 |

### Example C: In Vitro Human Transporter Efflux

### Example C1: MDR1 and BCRP

Madin-Darby canine kidney (MDCKII) cells expressing either MDR1 or BCRP are seeded onto Corning HTS Transwell® 96-well polycarbonate permeable (0.4 μm pore) supports at a density of 545,000 cells/cm². Cells are incubated for 4-8 days prior to assay, and monolayer integrity is assessed by measuring transepithelial electrical resistance (TEER). Test and reference compounds are diluted with the transport buffer (Hank's buffered salt solution (HBSS) HEPES pH 7.4) to concentrations of 10 and 1 μM, respectively. The final organic solvent concentration is 0.5% (v/v). Bidirectional (apical-to-basolateral and basolateral-to-apical) flux of the test and reference compounds is determined over a 2-hour incubation at 37° C. and 5% CO₂ with a relative humidity of 95%. At the end of the incubation, samples from the apical and basolateral side are taken and then precipitated with acetonitrile containing internal standard. After centrifugation at 3200×g, supernatants are diluted 1:1 (v/v) with water and subjected to analysis via HPLC-MS/MS. The integrity of the cell monolayers during the assay is confirmed by using the marker Lucifer yellow at a final concentration of 100 μM.

### Example C2: Caco-2

Following a 1-hr. equilibration period in cell culture medium maintained at 37° C. in an incubator containing a 5% CO₂ atmosphere at 95% relative humidity, Corning 96-well HTS Transwell® permeable support plates are seeded with 34,300 Caco-2 cells (ATCC) per well. Following seeding, plates are cultured for 14-18 days with cell culture medium replacement every other day beginning no later than 24 hr. after plating. Monolayer barrier integrity is monitored during the culture period using transepithelial electrical resistance (TEER) measurements taken with a Millicell Epithelial Volt-Ohm system. Caco-2 monolayers are considered ready for use when the TEER value >230 ohm·cm². Working stocks of individual test articles are prepared at a concentration of 5 μM by diluting 1 mM stocks prepared in DMSO into 10 mM HEPES. Prior to conducting the assay, Caco-2 plates are washed twice with prewarmed 10 mM HEPES and then equilibrated in 10 mM HEPES for 30 min. at 37° C. Following equilibration, test article flux is tested bidirectionally (in both the apical-to-basolateral and basolateral-to-apical directions) by adding test article working stocks to either the apical or basolateral chambers. Blank 10 mM HEPES is added to the opposite sides. Plates are then incubated at 37° C. for 2 hours. At the end of the incubation, aliquots from both sides of each monolayer are quenched with 4 volumes of ACN containing internal standards. Following a centrifugation step, supernatants are diluted with pure water prior to analysis via LC-MS/MS.

Apparent permeability (Pₐₚₚ, ×10⁻⁶ cm/s) is calculated for all transport assays using the following equation:

$${P}_{a p p} = \frac{dQ / dt}{A \times {D}_{0}}$$

Where: Pₐₚₚ is apparent permeability (×10⁻⁶ cm/s).
   dQ/dt is the rate of drug transport (pmol/s).
   A is the surface area of the membrane (cm²).
   D₀ is the initial donor concentration (nM; pmol/cm³).

Efflux ratio is determined by calculating the ratio of the Pₐₚₚ in the basolateral-to-apical direction to the Pₐₚₚ in the apical-to-basolateral direction using the following equation:

$$Efflux Ratio = \frac{{P}_{a p p ( B - to - A )}}{{P}_{a p p ( A - to - B )}}$$

Where: P_{app (B-to-A)} is the apparent permeability in the basolateral-to-apical direction.
   P_{app (A-to-B)} is the apparent permeability in the apical-to-basolateral direction.

### Example D: In Vivo Determination of Rat Kp,uu

### Determination of Fraction Unbound in Plasma (Pu)

The equilibrium dialysis method is used to investigate the in vitro binding of test articles and reference compounds to plasma proteins. Plasma samples containing 5 μM test article or blank dialysis buffer solution (phosphate buffered saline (PBS), pH 7.4) are added to separate chambers of the dialysis wells of the High Throughput equilibrium Dialysis (HTD) device. The dialysis plate is sealed and placed in an incubator at 37° C. with 5% CO₂ with shaking at approximately 100 rpm for 6 hours. All experiments are performed in duplicate. Ketoconazole (5 μM) is used as the reference compound. After incubation, the seal is removed, and 50 μL of post-dialysis samples are pipetted from both buffer and plasma chambers into fresh 96-well plates. Samples are equimatrilyzed by either addition of blank plasma to buffer samples or the addition of blank buffer to plasma samples. Subsequently, 400 μL (4 volumes) of acetonitrile containing internal are added to all samples to precipitate proteins prior to analysis by Ultra-high Performance Liquid Chromatography (UPLC)-MS/MS to determine the relative concentrations of test articles. The unbound fractions in plasma are calculated using the concentrations of test articles in buffer and plasma samples according to the following equation:

$$Percentage unbound ( \% ) = \frac{Analyte to IS Peak Area Ratio ( buffer chamber )}{Analyte to IS Peak Area Ratio ( plasma chamber )} \times 100$$

### Determination of Fraction Unbound in Brain Homogenate (Bu)

The equilibrium dialysis method is used to investigate the in vitro binding of test articles and reference compounds to rodent brain homogenate. Brains collected from naïve animals are weighed and homogenized in 4 volumes of PBS, pH 7.4. Brain homogenate samples containing 1 μM test article or blank dialysis buffer solution (PBS, pH 7.4) are added to separate chambers of the dialysis wells of the High Throughput equilibrium Dialysis (HTD) device. The dialysis plate is sealed and placed in an incubator at 37° C. with 5% CO₂ with shaking at approximately 100 rpm for 6 hours. All experiments are performed in duplicate. Telmisartan (5 μM) is used as the reference compound. After incubation, the seal is removed, and 50 μL of post-dialysis samples are pipetted from both buffer and brain homogenate chambers into fresh 96-well plates. Samples are equimatrilyzed by either addition of blank homogenate to buffer samples or the addition of blank buffer to homogenate samples. Subsequently, 400 μL (4 volumes) of acetonitrile containing internal are added to all samples to precipitate proteins prior to analysis by UPLC-MS/MS to determine the relative concentrations of test articles. The unbound fractions in diluted brain homogenate are calculated using the concentrations of test articles in buffer and homogenate samples according to the following equation:

$$Percentage unbound homogenate ( \% ) = \frac{Analyte to IS Peak Area Ratio ( buffer chamber )}{Analyte to IS Peak Area Ratio ( homogenate chamber )} \times 100$$

Correction to percentage unbound in undiluted brain is achieved with the following equation:

$$Percentage unbound brain ( \% ) = 100 \times \frac{1 / 5}{( \frac{1}{Percentage unbound homogenate / 100} - 1 ) + 1 / 5}$$

Determination of the Drug Brain-to-Plasma Partition Coefficient (Kp) and Drug Unbound Kp (Kp,uu) in Rat

Compounds are formulated either individually or in a cassette (as a mixture) at a concentration of 0.1 mg/mL/compound in sterile water containing 0.5% (w/v) methylcellulose 400 cP and administered to male Sprague-Dawley rats via oral gavage at a dose volume of 10 mL/kg. One animal is sacrificed at each of the following time points: 0.5, 1, 2, 4, 8, and 24 hours post-dose, and brain and blood samples are collected. Plasma is prepared from blood via refrigerated centrifugation, and plasma samples are stored frozen at −80° C. until bioanalysis. Brain samples are rinsed with saline to remove residual blood and blotted dry with a paper wipe. Brain samples are then weighed and homogenized with 3 volumes (v/w) of water and stored frozen at −80° C. until bioanalysis.

Prior to bioanalysis, plasma and brain samples are extracted with 4 volumes of acetonitrile containing internal standard and centrifuged for 15 minutes. Supernatants are diluted with 2 volumes of water and injected for analysis via HPLC-MS/MS. Plasma and brain homogenate drug concentrations are determined against calibration curves generated by spiking blank rat plasma or brain homogenate with drug across an appropriate concentration range. The brain homogenate concentration is corrected for the homogenization buffer dilution factor yielding total brain drug concentrations.

The brain-to-plasma partition coefficient (Kp) is determined for each compound, calculated as: AUCbrain:AUCplasma, provided tlast is identical in each matrix. If the drug concentration versus time profile for one matrix falls below the lower limit of quantification at a time point earlier than in the other matrix, then the brain Kp is calculated as the average of the ratios of total brain drug concentration to total plasma drug concentration measured at each time point where drug concentrations in both matrices are quantifiable.

The Kp, uu is then calculated from the Kp using the following equation: Kp,uu=Kp*(fraction unbound in brain homogenate/fraction unbound in plasma).

### Example E: HLM and hHEP Assays

### HLM

Working stocks of individual test articles are prepared at a concentration of 100 μM by diluting 10 mM stocks prepared in DMSO 100-fold (v:v) into ACN. Thawed hepatic microsomes are suspended in 100 mM potassium phosphate buffer, pH 7.4 to a microsomal protein concentration of 0.562 mg/mL. Diluted microsomes are combined with a solution of 10 mM NADPH, and the mixture is pre-warmed to 37° C. for 8 min. Reactions are initiated by addition of the test article working stocks to achieve a final test article concentration of 1 μM. Final microsomal protein and NADPH concentrations are 0.5 mg/mL and 1 mM, respectively. Incubations containing NADPH are run in duplicate. Test article losses mediated by non-CYP mechanisms are evaluated in a parallel set of incubations run in the absence of NADPH. Incubations lacking NADPH consist of 1 replicate per test article. Following incubation in a 37° C. water bath, aliquots of individual reactions are quenched with cold ACN containing internal standards at 0.5, 15-, 30-, 60-, 90-, and 120-min. Precipitated protein is pelleted via refrigerated centrifugation. Supernatants are diluted into an equal volume of pure water and mixed well prior to analysis via LC-MS/MS. In vitro intrinsic clearances (CLᵢₙₜ) in μL/min/mg are determined for each incubation from calculated in vitro half-lives determined using a standard log-linear regression approach. In vitro CLᵢₙₜ values are scaled up using the following physiological scaling factors: 40 mg microsomal protein/g human liver and 25.7 g human liver/kg body weight. Scaled intrinsic clearance values are finally introduced to the well-stirred liver model for the purpose of calculating predicted human hepatic clearance (CL_{hep,pred}) in mL/min/kg assuming a human liver blood flow of 21 mL/min/kg and making no corrections for test article binding to red blood cells, plasma proteins, or components of the incubation system.

hHEP

Working stocks of individual test articles are prepared at concentrations of 100 μM by diluting 10 mM stocks prepared in DMSO 100-fold (v:v) into ACN/H₂O (50/50, v:v). Human cryopreserved hepatocytes are thawed in a 37° C. water bath in <2 min., suspended in thawing media, and then centrifuged at 100×g for 10 min. Thawing media is aspirated, and pelleted hepatocytes are resuspended into incubation media at 1.5×10⁶ cells/mL. Cell viability is determined using an Acridine Orange/Propidium Iodine stain, and hepatocytes are further diluted with incubation media to 0.5×10⁶ viable cells/mL. Hepatocyte aliquots of 198 μL are added to wells of a 96-well plate, and test article incubations are initiated by the addition of 2 μL of 100 μM working stocks. Plates are incubated at 37° C. in a 5% CO₂ atmosphere at 95% relative humidity on an orbital shaker at 300 rpm. Incubations are performed in duplicate. Following incubation, aliquots of individual reactions are terminated by addition of ACN containing internal standards at 0, 30-, 60-, 90-, 120-, and 240-min. Precipitated protein is pelleted via refrigerated centrifugation. Supernatants are diluted into an equal volume of pure water and mixed well prior to analysis via LC-MS/MS. In vitro intrinsic clearances (CLᵢₙₜ) in μL/min/10⁶ cells are determined for each incubation from calculated in vitro half-lives determined using a standard log-linear regression approach. In vitro CLᵢₙₜ values are scaled up using the following physiological scaling factors: 99×10⁶ cells/g human liver and 25.7 g human liver/kg body weight. Scaled intrinsic clearance values are finally introduced to the well-stirred liver model for the purpose of calculating predicted human hepatic clearance (CL_{hep,pred}) in mL/min/kg assuming a human liver blood flow of 21 mL/min/kg and making no corrections for test article binding to red blood cells, plasma proteins, or components of the incubation system.

What is claimed is:

1. A compound which is: 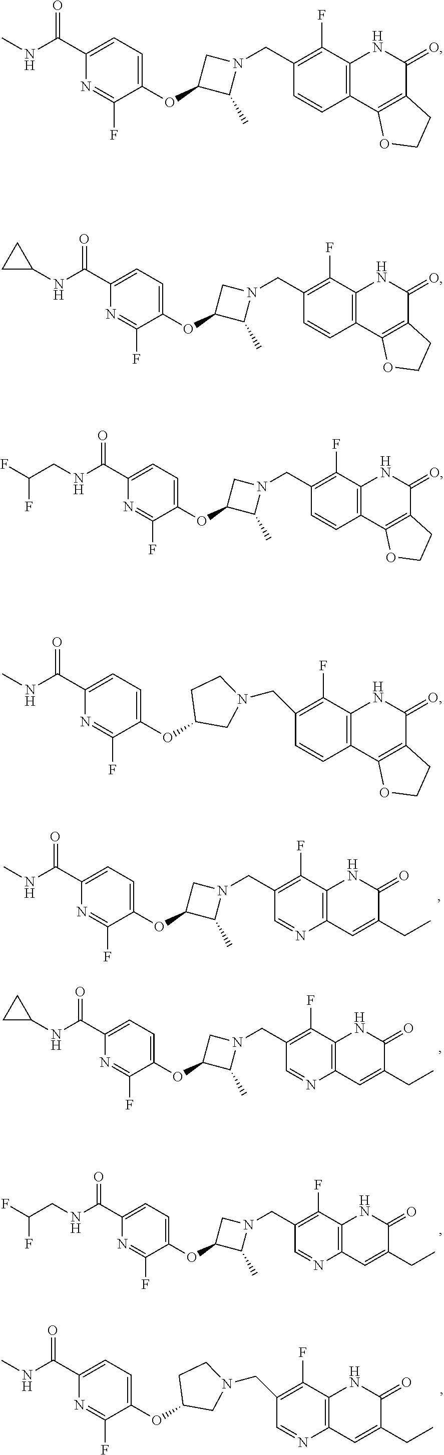 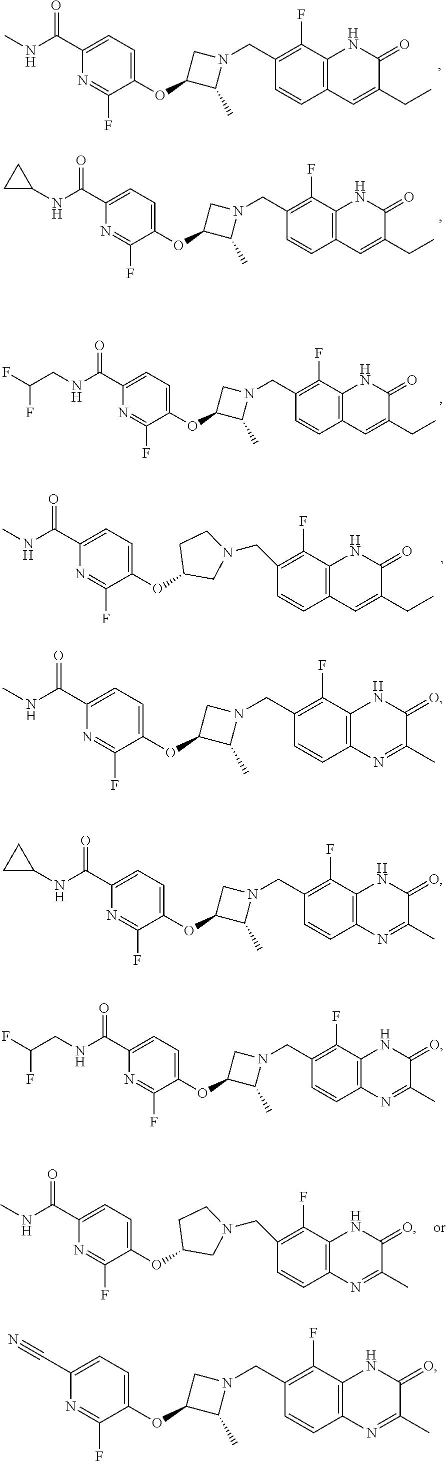
or a pharmaceutically acceptable salt thereof.

2. A pharmaceutical composition comprising a compound of claim 1, or a pharmaceutically acceptable salt thereof, and a pharmaceutically acceptable excipient.

3. A method of treating cancer in a subject in need thereof, the method comprising administering a therapeutically effective amount of a compound of claim 1, or a pharmaceutically acceptable salt thereof.

4. The method of claim 3, wherein the cancer is breast cancer, ovarian cancer, pancreatic cancer, prostate cancer, a hematological cancer, gastrointestinal cancer, or lung cancer.

5. The method of claim 3, wherein the cancer has metastasized in the brain.

6. A method of treating brain cancer in a subject in need thereof, the method comprising administering a therapeutically effective amount of a compound of claim 1, or a pharmaceutically acceptable salt thereof.

7. The method of claim 6, wherein the compound, or a pharmaceutically acceptable salt thereof, is brain penetrant.
